(19) United States
(12) Patent Application Publication
(10) Pub. No.: US 2008/0267964 A1
(43) Pub. Date: Oct. 30, 2008

(54) Synergistic Effect of Tgf-Beta Blockade and Immunogenic Agents on Tumors

(76) Inventors: Masaki Terabe, Bethesda, MD (US); Shun Takaku, Bethesda, MD (US); Jay A. Berzofsky, Bethesda, MD (US)

Correspondence Address: KLARQUIST SPARKMAN, LLP, 121 S.W. SALMON STREET, SUITE #1600, PORTLAND, OR, 97204-2988, US

(21) Appl. No.: 11/816,410

(22) PCT Filed: Feb. 16, 2006
(86) PCT No.: PCT/US06/05888

§ 371 Date: Aug. 15, 2007

Related U.S. Application Data
(60) Provisional application No. 60/654,329, filed on Feb. 17, 2005.

(51) Int. Cl. A61K 39/395; A61P 31/00

(52) U.S. Cl. 424/138.1

(57) ABSTRACT

Methods are provided herein for synergistically affecting tumor growth in a subject, involving the administration to the subject of an agent that blocks the TGF-β signaling pathway in combination with an immunogenic agent. The agent that blocks the TGF-β signaling pathway is believed to inhibit the immunosuppressive effects of TGF-β, while the immunogenic agent is believed to enhance an immune response. Surprisingly, the combination of such elements produces a synergistic effect. In one embodiment, the administration of the 1D11.16 anti-TGF-β antibody in combination with the human papilloma virus E7₍₄₉₋₅₇₎ peptide enhances tumor regression and tumor-specific CTL response in the subject. In another embodiment, the administration of the 1D11.16 anti-TGF-β antibody in combination with irradiated CT26 cells enhances tumor regression in the subject. The method of administering the combination of agents to the subject is more effective than the administration of each agent individually, or the sum of their individual effects.

### REFERENCE TO RELATED APPLICATIONS

This application claims the benefit of U.S. Provisional Application No. 60/654,329, filed Feb. 17, 2005, the contents of which are hereby incorporated by reference.

### FIELD

The present disclosure is related to methods of affecting tumors. More specifically, the disclosure relates to the synergistic effects of blocking transforming growth factor (TGF)-β signaling, combined with the administration of immunogenic agents, in order to inhibit tumor growth.

### BACKGROUND

Transforming growth factor (TGF)-β and its receptors are expressed in essentially all tissues, and have been found to be important in many cellular processes. TGF-β has been shown to play a role in cell growth and differentiation, immunosuppression, inflammation, and the expression of extracellular matrix proteins. For example, TGF-β inhibits the growth of many cell types, including epithelial cells, but it also has been shown to stimulate the proliferation of various types of mesenchymal cells. In animal models, TGF-β has been shown to attenuate the symptoms associated with various diseases and disorders, including rheumatoid arthritis, multiple sclerosis, wound healing, bronchial asthma, and inflammatory bowel disease. In the clinical setting, it has been used to enhance wound healing. TGF-β also has many immunoregulatory functions, including modulation of T-cell proliferation, apoptosis, activation and differentiation.

TGF-β is expressed in high amounts in many tumors and is known to have at least two important roles in cancer (see, for instance, U.S. Pat. No. 6,046,165). Since TGF-β is generally growth inhibitory, under-expression of TGF-β, activating mutations in the TGF-β receptor, or activating mutations of any of the downstream targets of TGF-β can result in uncontrolled proliferation. However, TGF-β is also highly immunosuppressive. Tumor cells that are no longer responsive to the growth inhibitory effects of TGF-β up-regulate the expression of TGF-β to protect themselves from the immune system and thereby escape immunosurveillance (Mule et al., Cancer Immunol Immunother 26(2):95-100, 1988; Gorelik and Flavell, Nature Medicine 7(10):1118-1122, 2001).

The inhibition of TGF-B signaling has been shown to have an inhibitory effect on tumor growth. For example, Gorelik and Flavell (Nature Medicine 7(10):1118-1122, 2001) demonstrated that a blockade of TGF-β signaling allowed the generation of an immune response capable of rejecting tumors in mice that had been challenged with live tumor cells. Also, U.S. patent application Ser. No. 10/176,266 indicates that soluble TGF-β antagonists (such as anti-TGF-β antibodies) are capable of suppressing metastasis. In addition, Terabe et al. (J. Exp. Med. 198: 1741-1752, 2003) demonstrated that treatment of tumor-bearing mice with anti-TGF-β monoclonal antibodies could prevent tumor recurrence and reduce the number of tumor lung metastases.

Vaccines that elicit cellular immune responses also have been used to treat or control the growth of tumors that have evaded immunosurveillance. For example, antigen presenting cells, such as dendritic cells (DCs), have been used in vaccines to present tumor-specific antigens in order to stimulate CD8⁺ cytotoxic T lymphocytes (CTLs) (Okada et al., Int. J Cancer 78:196-201, 1998). Alternatively, subjects can be vaccinated with irradiated, whole tumor cells obtained from the subject, in order to stimulate a CTL immune response (PCT Patent Application No. PCT/US97/10540). However, such vaccines have demonstrated limited success. Thus, there is a continuing need to develop new methods of preventing and/or treating tumors.

### SUMMARY

This disclosure provides methods of synergistically affecting malignant neoplasm in a subject, for instance specifically enhancing tumor regression in a subject. In a representative example of the methods, a subject is administered a therapeutically effective amount of a combination of at least two agents. A first agent in the combination is believed to induce and/or enhance an immune response. By way of example, the agent which induces and/or enhances an immune response in some instances is a peptide; in other instances, it is an inactivated whole cell. A second agent in the combination is believed to block the TGF-β signaling pathway and inhibit the immunosuppressive effects of TGF-β. By way of example, the agent which blocks the TGF-β signaling pathway in some instances is an antibody which binds TGF-β. In other embodiments, the agent which blocks the TGF-13 signaling pathway is an antibody which binds the TGF-β receptor or a downstream signaling molecule in the TGF-β pathway. In yet other embodiments, the TGF-β blockade agent is a soluble form of a TGF-β receptor, or a fusion protein comprising such, or any other molecule capable of blocking a function or activity of the TGF-β signaling pathway.

The foregoing and other features and advantages will become more apparent from the following detailed description of several embodiments, which proceeds with reference to the accompanying figures.

### BRIEF DESCRIPTION OF THE FIGURES

FIG. 1 is a graph illustrating that the blockade of TGF-β synergistically enhances vaccine efficacy. C57BL/6 mice were inoculated subcutaneously with 2×10⁴ TC1 cells. On day four, some mice were immunized subcutaneously with 100 μg of Human Papilloma Virus (HPV)16 E7₍₄₉₋₅₇₎ peptide emulsified in incomplete Freund's adjuvant with a hepatitis B virus (HBV) core helper epitope peptide (50 nmol) and granulocyte-macrophage colony stimulating factor (GM-CSF; 5 μg) (filled squares and filled circles). Some mice were injected with 100 μg of anti-TGF-β monoclonal antibody (1D11.16) intraperitoneally three times a week from the day of tumor inoculation (open triangles) or from day four (inverted triangles and filled squares) until the end of the experiment. Five mice were used for each group.

FIGS. 2A and 2B are a series of graphs illustrating the frequency of tumor-antigen specific CD8⁺ T cells and the tumor-antigen specific IFN-γ production by CD8⁺ T cells induced by the HPV E7₍₄₉₋₅₇₎ peptide vaccine. C57BL/6 mice were inoculated subcutaneously with 2×10⁴ TC1 cells. On day four, some mice were immunized subcutaneously with 100 μg of HPVL6 E7₍₄₉₋₅₇₎ peptide emulsified in incomplete Freund's adjuvant with a hepatitis B virus (HBV) core helper epitope peptide (50 nmol) and GM-CSF (5 μg; filled triangles). Some mice were injected with 100 μg of anti-TGF-β monoclonal antibody (1D11.16) intraperitoneally three times a week from day four until the end of the experiment (filled squares). Two weeks after immunization, the mice were euthanized and spleen cells were examined for a specific response against HPV E7₍₄₉₋₅₇₎. To measure the number of HPV E7₍₄₉₋₅₇₎-specific CD8⁺ T cells, spleen cells were stained with Db-tetramer loaded with HPV E7₍₄₉₋₅₇₎ peptide along with anti-mouse CD8 antibody, and measured by flow cytometry (FIG. 2A). For measurement of a HPV E7₍₄₉₋₅₇₎-specific IFN-y producing response of CD8⁺ T cells, the cells were cultured with T cell-depleted naïve spleen cells pulsed with or without 0.1 μM of HPV E7₍₄₉₋₅₇₎ overnight. Then the cells were stained for surface CD8 and intracellular IFN-y, and measured by flow cytometry (FIG. 2B).

FIG. 3 is a graph illustrating the in vivo tumor antigen-specific lytic activity induced by the HPV E7₍₄₉₋₅₇₎ peptide vaccine. C57BL/6 mice were inoculated subcutaneously with 2×10⁴ TC1 cells. On day four, some mice were immunized subcutaneously with 100 μg of HPV16 E7₍₄₉₋₅₇₎ peptide emulsified in incomplete Freund's adjuvant with a hepatitis B virus (HBV) core helper epitope peptide (50 nmol) and GM-CSF (5 μg). Some mice were injected with 100 μg of anti-TGF-β monoclonal antibody (1D11.16) intraperitoneally three times a week from day four until the end of the experiment. Thirteen days after immunization of TC1-challenged mice, a 1:1 mixture of spleen cells (1×10⁷ of each) of naïve mice pulsed with or without 0.1 μM of HPV E7₍₄₉₋₅₇₎ and labeled with different concentrations of carboxy-fluorescein diacetate, succinimidyl ester (CFSE) was injected intravenously. The next day, spleen cells from the mice were harvested and residual CFSE cells were measured by flow cytometry. The proportion of the cells with different CFSE brightness was determined, and compared with the proportion in naïve cells that received the same cells to compute HPV E7₍₄₉₋₅₇₎-specific lytic activity.

FIG. 4 is a graph illustrating that the protection induced by the HPV E7₍₄₉₋₅₇₎ peptide vaccine is mediated by CD8⁺ cytotoxic T lymphocytes (CTLs). C57BL/6 mice were inoculated subcutaneously with 2×10⁴ TC1 cells. On day 7, some mice were immunized subcutaneously with 100 μg of HPV E7₍₄₉₋₅₇₎ peptide emulsified in incomplete Freund's adjuvant with a HBV core helper epitope peptide (50 nmol) and GM-CSF (5 μg) (squares and circles). Some mice were injected with 100 μg of anti-TGF-β monoclonal antibody (1D11.16) intraperitoneally three times a week from day 7 to day 21 (squares) or with a control antibody 13C4 (circles). Some mice were also treated intraperitoneally with 0.5 mg of anti-CD8 monoclonal antibody (2.43) on days 7, 8, 13, 15, 20 (triangles, open circles and open squares). Five mice were used for each group.

FIG. 5 is a graph illustrating that blockade of TGF-β synergistically enhances the protective efficacy of a whole cell vaccine in mice. BALB/c mice were vaccinated with 1×10⁵ irradiated (25,000 rad) CT26 cells subcutaneously. Some vaccinated or unvaccinated mice were treated with 200 μg (at the time of vaccination and CT26 challenge) or 100 μg (other time points) anti-TGF-β monoclonal antibody (1D11.16) or control antibody (13C4) intraperitoneally (ip) three times a week from the time of vaccination to two weeks after CT26 challenge. Three weeks after vaccination, the mice were challenged with 1×10⁶ live CT26 cells subcutaneously. One and two days before, and 4, 7, 10, and 14 days after CT26 challenge, some vaccinated mice treated with 1D11 were also treated with anti-CD8 monoclonal antibody (2.43) to show the CD8 dependence of the protection. Tumors were measured by a caliper gage, and tumor size was determined as the product of tumor length (mm)×tumor width (mm). Five female BALB/c mice were used for each group.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. Sequences are referred to herein as follows:

SEQ ID NO: 1 is the amino acid sequence of the E7₍₄₉₋₅₇₎ peptide (RAHYNIVTF).

SEQ ID NO: 2 is the amino acid sequence of the complete E7 polypeptide (MHGDTPTLHEYMLDLQPETTDLYCYEQLNDSSEEEDEIDGPAGQAEPDRA HYNIVTFCCKCDSTLRLCVQSTHVDIRTLEDLLMGTLGIVCPICSQKP).

SEQ ID NO: 3 is the amino acid sequence of the AH1 peptide (SPSYVYHQF).

SEQ ID NO: 4 is the amino acid sequence of gp 100₂₀₉₋₂₁₇ (ITQVPFSV).

SEQ ID NOs: 5 and 6 are the amino acid sequences of two TARP-derived peptides (FLRNFSLM and FVFLRNFSL, respectively).

### DETAILED DESCRIPTION

### I. Abbreviations

APC antigen presenting cell

CTL cytotoxic T lymphocyte

DC dendritic cell

GM-CSF granulocyte-macrophage colony stimulating factor

HBV hepatitis B virus

HPV human papilloma virus

IFN interferon

IL interleukin

NK cells natural killer cells

TGF transforming growth factor

TNF tumor necrosis factor

### II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments, the following explanations of specific terms are provided:

Activity of a TGF-β receptor expressing immune cell: A biological activity of a cell that expresses a TGF-β receptor. The biological activity of such a cell can include target cell lysis, cell proliferation, cytokine production, inhibition of growth of a tumor or other malignant neoplasm, inhibition of tumor recurrence or recurrence of another malignant neoplasm, or inhibition of malignant neoplasm metastasis, such as tumor metastasis. A change in activity of a cell that expresses a TGF-β receptor, such as a reduction in target cell lysis, cytokine production, inhibition of tumor recurrence, or inhibition of tumor metastasis, can result from a blockade of TGF-β signaling. A cell activity can be measured by any method known to one of skill in the art. For example, the ability to lyse a target cell can be measured by a chromium (Cr) release assay, which is well known to those of ordinary skill in the art. In another example, the ability to produce cytokines can be measured by western blot, ELISA, intracellular cytokine staining, ELISPOT, or northern analysis. In yet another example, the ability to enhance tumor (or malignant neoplasm) regression, inhibit tumor (or malignant neoplasm) recurrence, or inhibit tumor (or malignant neoplasm) metastasis can be measured by the number of mice with tumors following treatment (for example, following administration of a combination therapy including an anti-TGF-β antibody) versus control mice.

Adjuvant: A substance that non-specifically enhances the immune response to an antigen. Development of adjuvants for use in humans is reviewed in Singh et al., Nat. Biotechnol. 17:1075-1081, 1999, which discloses that, at the time of its publication, aluminum salts and the MF59 microemulsion were the only vaccine adjuvants approved for human use.

Affecting tumor (or malignant neoplasm) growth: Having an impact, particularly a negative impact, on growth of a tumor (or growth or development of any malignant neoplasm), for instance by inhibiting, preventing or reversing tumor growth or development. Affecting tumor growth includes preventing further growth of an existing tumor, enhancing tumor regression, inhibiting tumor recurrence, or inhibiting tumor metastasis. An agent that blocks the TGF-β signaling pathway, such as a neutralizing agent or an enzyme, can affect tumor growth. Similarly, an immunogenic agent, such as a tumor peptide antigen or an inactivated whole cell, can affect tumor growth.

Agent: Any substance, including, but not limited to, an antibody, antagonist, chemical compound, small molecule, peptide mimetic, peptide, polypeptide, lysed cell or whole cell. An agent can be produced by a subject's body. In one embodiment, an agent enhances anti-tumor immunity. In other embodiments, an agent prevents further growth of an existing tumor, enhances tumor regression, inhibits tumor recurrence, or inhibits tumor metastasis. An agent that blocks the TGF-β signaling pathway can be a protein, such as an enzyme or an antibody, that inhibits (neutralizes) the function of a protein in the TGF-β signaling pathway (for example, TGF-p). In one embodiment, an agent blocks the immunosuppressive effects of TGF-β by neutralizing an activity of TGF-β. An immunogenic agent is an agent that induces and/or enhances an immune response.

Animal: Living multi-cellular vertebrate organisms, a category that includes, for example, mammals and birds. The term mammal includes both human and non-human mammals. Similarly, the term “subject” includes both human and veterinary subjects.

Antibody: Immunoglobulin (Ig) molecules and immunologically active portions of Ig molecules, for instance, molecules that contain an antigen binding site which specifically binds (immunoreacts with) an antigen. In one embodiment the antigen is TGF-β. In other embodiments, the antigen is the TGF-β receptor or a TGF-β downstream signaling molecules (for example, Smad2, Smad3, Smad4, Smad complex DNA-binding co-factors). Monoclonal, polyclonal, and humanized immunoglobulins are encompassed by the disclosure. The disclosure also includes synthetic and genetically engineered variants of these immunoglobulins.

Humanized antibodies include genetically engineered antibodies designed to transfer the specificity of a non-human antibody to a human immunoglobulin by exchange of specific or critical non-human residues. A humanized antibody can include a human framework region and one or more complementarity determining regions (CDRs) from a non-human (such as a mouse, rat, or synthetic non-human) immunoglobulin (U.S. Pat. No. 6,495,137, U.S. Pat. No. 6,818,749). In one embodiment, the DNA encoding hypervariable loops of mouse monoclonal antibodies or variable regions selected in phage display libraries is inserted into the framework regions of human Ig genes. In another embodiment, murine residues important in antigen binding (ligand contact residues or specificity determining residues (SDRs), or essential framework residues) are inserted into the corresponding position of the variable region of a human Ig sequence. In yet another embodiment, a human residue is inserted into the corresponding position of a murine Ig sequence. Antibodies can be “customized” to have a desired binding affinity or to be minimally immunogenic in the humans treated with them.

A naturally occurring antibody (for example, IgG) includes four polypeptide chains, two heavy (H) chains and two light (L) chains inter-connected by disulfide bonds. However, it has been shown that the antigen-binding function of an antibody can be performed by fragments of a naturally occurring antibody. Thus, these antigen-binding fragments are also intended to be designated by the term “antibody”. Examples of binding fragments encompassed within the term antibody include (i) an Fab fragment consisting of the VL, VH, CL and CH1 domains; (ii) an Fd fragment consisting of the VH and CH1 domains; (iii) an Fv fragment consisting of the VL and VH domains of a single arm of an antibody, (iv) a dAb fragment (Ward et al., Nature 341:544, 1989) which consists of a VH domain; and (v) an F(ab′)₂ fragment, a bivalent fragment comprising two Fab fragments linked by a disulfide bridge at the hinge region.

Furthermore, although the two domains of the Fv fragment are coded for by separate genes, a synthetic linker can be made that enables them to be made as a single protein chain (known as single chain Fv (scFv); Bird et al. Science 242:423, 1988; and Huston et al. Proc. Natl. Acad. Sci. 85:5879, 1988) by recombinant methods. Such single chain antibodies, as well as dsFv, a disulfide stabilized Fv (Bera et al. J. Mol. Biol 281:475-483, 1998), and dimeric Fvs (diabodies), that are generated by pairing different polypeptide chains (Holliger et al. Proc. Natl. Acad. Sci. 90:6444-6448. 1993), are also included.

In one embodiment, antibody fragments for use in this disclosure are those which are capable of cross-linking their target antigen, for example, bivalent fragments such as F(ab′)₂ fragments. Alternatively, an antibody fragment which does not itself cross-link its target antigen (for example, a Fab fragment) can be used in conjunction with a secondary antibody which serves to cross-link the antibody fragment, thereby cross-linking the target antigen. Antibodies can be fragmented using conventional techniques and the fragments screened for utility in the same manner as described for whole antibodies. An antibody is further intended to include humanized monoclonal molecules that specifically bind the target antigen.

“Specifically binds” refers to the ability of individual antibodies to specifically immunoreact with an antigen. This binding is a non-random binding reaction between an antibody molecule and the antigen. In one embodiment, an antigen is a TGF-β. Binding specificity is typically determined from the reference point of the ability of the antibody to differentially bind the antigen of interest and an unrelated antigen, and therefore distinguish between two different antigens, particularly where the two antigens have unique epitopes. An antibody that specifically binds to a particular epitope is referred to as a “specific antibody.” In one embodiment, the monoclonal antibody obtained from hybridoma 1D11.16 (ATCC Accession No. HB 9849) binds TGF-β and therefore is specific. In another embodiment, the human monoclonal antibody GC 1008 (Genzyme Corp., Cambridge, Mass.), with similar pan-anti-TGF-β specificity as the 1D1.16 antibody, is used.

Antigen: Any molecule that is specifically bound by an antibody or recognized by a T-lymphocyte antigen receptor. An antigen is also a substance that antagonizes or stimulates the immune system to produce antibodies or T-cell responses, for example an antigen on the surface of an antigen-presenting cell. Antigens are often found on substances (such as allergens, bacteria, or viruses) that invade the body.

In one embodiment an antigen is a TGF-β. In other embodiments, the antigen is the TGF-β receptor or a TGF-β downstream signaling molecules (for example, Smad2, Smad3, Smad4, or Smad complex DNA-binding co-factors).

Carrier: An immunogenic macromolecule to which an antigenic but not highly immunogenic molecule, for example a tumor peptide, can be bound. When bound to a carrier, the bound molecule becomes more immunogenic. Carriers are chosen to increase the immunogenicity of the bound molecule and/or to elicit antibodies against the carrier which are diagnostically, analytically, and/or therapeutically beneficial. Covalent linking of a molecule to a carrier confers enhanced immunogenicity and T-cell dependence (Pozsgay et al., PNAS 96:5194-97, 1999; Lee et al., J. Immunol. 116:1711-18, 1976; Dintzis et al., PNAS 73:3671-75, 1976). Useful carriers include polymeric carriers, which can be natural (for example, polysaccharides, polypeptides or proteins from bacteria or viruses), semi-synthetic or synthetic materials containing one or more functional groups to which a reactant moiety can be attached.

Examples of bacterial products for use as carriers include bacterial toxins, such as B. anthracis PA (including fragments that contain at least one antigenic epitope and analogs or derivatives capable of eliciting an immune response), LF and LeTx, and other bacterial toxins and toxoids, such as tetanus toxin/toxoid, diphtheria toxin/toxoid, P. aeruginosa exotoxin/toxoid/, pertussis toxin/toxoid, and C. perfringens exotoxin/toxoid. Viral proteins, such as hepatitis B surface antigen and core antigen can also be used as carriers, as well as proteins from higher organisms such as keyhole limpet hemocyanin, horseshoe crab hemocyanin, edestin, mammalian serum albumins, and mammalian immunoglobulins. Additional bacterial products for use as carriers include bacterial wall proteins and other products (for example, streptococcal or staphylococcal cell walls and lipopolysaccharide (LPS)).

Covalent Bond: An interatomic bond between two atoms, characterized by the sharing of one or more pairs of electrons by the atoms. The terms “covalently bound,” “covalently linked,” or “covalently fused” refer to making two separate molecules into one contiguous molecule. The terms include reference to joining a tumor peptide or polypeptide directly to a carrier molecule, and to joining a tumor peptide or polypeptide indirectly to a carrier molecule, with an intervening linker molecule.

Cytokines: Proteins, made by cells, that mediate inflammatory and immune reactions. In one embodiment, a cytokine is a chemokine, a molecule that affects cell movement. Cytokines include, but are not limited to, interleukins (for example, interleukin (IL)-4, IL-8, IL-10, IL-13), granulocyte-macrophage colony stimulating-factor (GM-CSF), neurokinin, tumor necrosis factors (TNFs) (for example, TNF-α, TNF-β), interferons (IFNs) (for example, IFN-α, IFN-β, IFN-γ) and TGF-βs (for example, TGF-β-1, TGF-β-2).

Cytotoxic T lymphocyte (CTL): A lymphocyte that is able to kill either self cells presenting foreign antigens, or abnormal self cells, including tumor cells, marked for destruction by the cellular immune system. CTLs can destroy cells infected with viruses, fungi, parasites, or certain bacteria. CTLs usually express the CD8 cell surface marker and recognize peptides displayed by class I major histocompatibility complex (MHC) molecules. CTLs kill virus-infected cells and tumor cells, whereas antibodies generally target free-floating viruses or bacteria in the blood. CTL killing of infected cells involves the release of cytoplasmic granules whose contents include membrane pore-forming proteins and enzymes. CTLs perform an immune surveillance function by recognizing and killing potentially malignant cells that express peptides that are derived from mutant cellular proteins or oncogenic viral proteins and are presented in association with class I MHC molecules. CTL-mediated tumor immunosurveillance is down-regulated by TGF-β as disclosed herein.

CTL assay: Activated CTLs generally kill any cells that display the specific peptide:MHC class I complex they recognize. CTL activity can be determined by using an assay that measures the ability of a CTL to kill a target cell (a cell expressing a specific peptide:MHC class I complex). A classical assay for CTL activity is the chromium release assay (WO 2004/037209, incorporated herein by reference). Target cells expressing an antigen on their surface are labeled with a radioactive isotope of chromium (⁵¹Cr). CTLs of a subject are then mixed with the target cell and incubated for several hours. Lysis of antigen-expressing cells by CTLs releases ⁵¹Cr into the medium which can be detected and quantified. The ability of CTLs to cause antigen-specific lysis is calculated by comparing lysis (correlated with chromium release) of target cells expressing the antigen or control antigens in the presence or absence of effector cells, and is usually expressed as the percent antigen-specific lysis.

E7₍₄₉₋₅₇₎ peptide: A nine amino acid long portion of the human papilloma virus E7 polypeptide (SEQ ID NO: 2). The E7₍₄₉₋₅₇₎ peptide (SEQ ID NO: 1) has a defined, CTL-recognized, MHC class I-restricted peptide epitope and induces a strong CTL response in vivo.

Epitope: A site on an antigen recognized by an antibody or T cell. These are particular chemical groups or contiguous or non-contiguous peptide sequences on a molecule that are antigenic, that is, that elicit a specific immune response. An antibody binds a particular antigenic epitope based on the three dimensional structure of the antibody and the matching (or cognate) epitope. Epitopes are also called antigenic determinants.

Immune cell: Any cell involved in a host defense mechanism. These include, for example, T cells, B cells, natural killer (NK) cells, NKT cells, neutrophils, mast cells, macrophages, antigen-presenting cells, basophils, eosinophils, and neutrophils.

Immune response: A collective and coordinated response to the introduction of a foreign (for example, non-self) substance in a subject, which response is mediated by the cells and molecules of the immune system. One example of an immune response is CTL-mediated tumor immunosurveillance. Another example of an immune response is one that is specific for a particular antigen (an “antigen-specific response”), such as a tumor-specific antigen (for example an isolated tumor peptide or the tumor peptides expressed in or on a whole, intact cell). Yet another example of an immune response is one that is stimulated by the presence of a cytokine. An immune response can be prophylactic or therapeutic.

Immunogenic agent: An agent that has a stimulatory effect on at least one component of the immune response, thereby causing or enhancing an immune response. Examples of immunogenic agent include nucleic acid sequences, tumor peptide antigens, and inactivated whole cells, though other immunogenic agents are known to those skilled in the art. In some embodiments, the immune response provides protective immunity, in that it enables the subject to prevent the establishment of a tumor, inhibit further growth of an existing tumor, or reduce the size of an existing tumor, for instance. Without wishing to be bound by a particular theory, it is believed that an immunogenic response may arise from the generation of neutralizing antibodies, T-helper, or cytotoxic cells of the immune system, or all of the above. In some instances, an immunogenic agent is referred to as a vaccine, for example a tumor vaccine, a peptide vaccine, a whole cell vaccine, a DNA vaccine, or a vector vaccine.

In some embodiments, an “effective amount” or “immune-stimulatory amount” of an immunogenic agent, or a composition including an immunogenic agent, is an amount which, when administered to a subject, is sufficient to engender a detectable immune response. Such a response may comprise, for instance, generation of an antibody specific to one or more of the epitopes provided by the immunogenic agent. Alternatively, the response may comprise a T-helper or CTL-based response to one or more of the epitopes provided by the immunogenic agent. All three of these responses may originate from naïve or memory cells. In other embodiments, a “protective effective amount” of an immunogenic agent, or a composition including an immunogenic agent, is an amount which, when administered to a subject, is sufficient to confer protective immunity upon the subject. In further embodiments, a “therapeutic effective amount” of an immunogenic agent, or a composition including an immunogenic agent, is an amount which, when administered to a subject, is sufficient to confer therapeutic immunity upon the subject.

Immunosuppression: Inhibition of one or more components of the adaptive or innate immune system as a result of an underlying disease, or intentionally induced by drugs for the purpose of preventing or treating graft rejection or autoimmune disease (in Cellular and Molecular Immunology, fourth edition, WB Saunders Co., 2000).

Immunosuppressive agent: An agent that has an inhibitory effect on at least one function of the immune response thereby causing immunosuppression. One example of an immunosuppressive agent is TGF-β. An immunosuppressive agent can prevent the immune system from reacting to foreign (non-self) substances and fighting disease, such as a tumor or other abnormal growth.

TGF-β is highly immunosuppressive as illustrated by the fact that CD8⁺ CTL-mediated tumor immunosurveillance is down-regulated by TGF-β. It has been proposed that TGF-β is involved in tumor “escape.” Tumor cells that are no longer responsive to the growth-inhibitory effects of TGF-β up-regulate the expression of TGF-β to protect themselves from the immune system and thereby escape immunosurveillance (Mule et al, Cancer Immunol Immunother 26:95, 1988; Gorelik and Flavell, Nature Medicine 7:1118, 2001).

The mechanisms of down-regulation of tumor immunosurveillance and immunosuppression by TGF-β can be studied, for instance, using a mouse tumor model in which tumors show a “growth-regression-recurrence” pattern following tumor inoculation in the mouse.

Immunosurveillance: Function of the immune system to recognize and destroy cells that express a foreign antigen (for example, tumor or microbial antigens). In one embodiment, immunosurveillance is the function of T lymphocytes to recognize and destroy transformed cells before they grow into tumors, and to kill tumors after they are formed. One specific, non-limiting example of immunosurveillance is CD8⁺ CTL-mediated tumor immunosurveillance.

Isolated: An “isolated” biological component (such as a nucleic acid molecule, protein or organelle) has been substantially separated or purified away from other biological components in the cell of the organism in which the component naturally occurs, for instance, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell, as well as chemically synthesized biopolymers. The terms “isolated” does not require absolute isolation. Similarly, the term “substantially separated” does not require absolute separation.

Lymphocytes: A type of white blood cell that is involved in the immune response of the body. There are two main classes of lymphocytes: B-cells and T-cells. A third class of lymphocytes is Natural Killer (NK) cells. Cytotoxic T lymphocytes (CTL) and NKT cells are types of T cells.

Mammal: This term includes both human and non-human mammals. Similarly, the term “subject” includes both human and veterinary subjects.

Metastasis: The spread of a tumor from one part of the body to another. Tumors formed from cells that have spread are called “secondary tumors” and contain cells that are like those in the original (primary) tumor. Metastasis is caused by at least a single tumor cell that is derived from an original tumor and that circulates or migrates to a different site from the original tumor. Metastasis requires the establishment of a new blood supply at the new tumor site.

Natural Killer (NK) cells: A type of lymphocyte (neither a T cell nor a B cell) that does not express the CD3 cell surface marker and does not use a conventional T cell receptor or B cell receptor to recognize its target. NK cells have activating or inhibitory receptors that detect the presence or absence of MHC molecules on target cells but, unlike T cell receptors, these are not antigen specific or MHC restricted.

NK cells provide part of the innate immune defense against virus-infected cells and cancer cells that is nonspecific. They do not have memory and are not induced by immunization with specific antigen. NK cells can mediate antibody-dependent cellular cytotoxicity (ADCC) through their Fc receptors. In the mouse, they have been identified by a surface marker called NK1.1, but are negative for the T cell markers CD3, CD4, and CD8. Subjects with immunodeficiencies, such as those caused by HIV infection, often have a decrease in “natural” killer cell activity.

Neutralize: Descriptive of an agent that can inhibit the activity of a molecule. Examples of a neutralizable molecule include TGF-β, the TGF-β receptor, or a TGF-β downstream signaling molecule. In one embodiment, neutralizing TGF-β inhibits the TGF-β signaling pathway, thereby inhibiting the immunosuppressive effects of TGF-p. Agents are disclosed herein to neutralize an activity of a molecule, for instance by any measure amount. The term “neutralize” does not require absolute neutralization. Similarly, the term “inhibits” does not require absolute inhibition.

By way of example, an agent can neutralize a molecule by specifically binding it, thereby preventing the molecule from performing its function or one of its functions. In one embodiment, the neutralizing agent prevents a molecule from interacting with other molecules, for example by preventing TGF-β from interacting with the TGF-β receptor, thereby neutralizing an activity of TGF-p. One specific, non-limiting example of a neutralizing agent is the 1D11.16 anti-TGF-β monoclonal antibody. Another example is the GC1008 human monoclonal anti-TGF-β antibody (Genzyme Corp., Cambridge, Mass.).

NKT cells: T cells that express the CD3 cell surface marker and have a conventional type of alpha-beta T cell receptor, but the repertoire of the alpha-beta T cell receptor is limited, so that most NKT cells recognize a glycolipid antigen presented by the non-classical class I MHC molecule CD1d. CD1d molecules are MHC (major histocompatibility complex) class I-like molecules that present glycolipids, rather than peptides, to T lymphocytes. The majority of NKT cells use a limited repertoire of T cell receptors, especially the V-alpha 14/V-beta 8 pair in the mouse and the V-alpha 24 in the human. They have the ability to kill target cells, but one of their major functions is to secrete cytokines very early in an immune response. They all express CD3, and some express CD4, whereas some are CD4/CD8 double negative. They were originally described as NKT cells in the mouse because they express the NK1.1 marker, like NK cells, but that is their only similarity with NK cells. They are now more commonly defined as T cells that are CD1d restricted.

Nucleotide: This term includes, but is not necessarily limited to, a monomer that includes a base linked to a sugar, such as a pyrimidine, purine or synthetic analogs thereof, or a base linked to an amino acid, as in a peptide nucleic acid (PNA). A nucleotide is one monomer in a polynucleotide. The term also includes other art-obvious modifications of such molecules that can form part of a polynucleotide. A nucleotide sequence refers to the sequence of bases in a polynucleotide.

Parenteral: Administered outside of the intestine, for example, not via the alimentary tract. Generally, parenteral formulations are those that will be administered through any possible mode except ingestion. This term especially refers to injections, whether administered intravenously, intrathecally, intramuscularly, intraperitoneally, or subcutaneously, and various surface applications including intranasal, intradermal, and topical application, for instance.

Peptide: Any compound containing two or more amino-acid residues joined by amide bonds, formed from the carboxyl group of one residue and the amino group of the next. The broad term “peptide” includes oligopeptides, polypeptides, and proteins.

Pharmaceutically acceptable carriers: The pharmaceutically acceptable carriers useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polypeptide: A polymer in which the monomers are amino acid residues that are joined together through amide bonds. When the amino acids are alpha-amino acids, either the L-optical isomer or the D-optical isomer can be used, the L-isomers being preferred in nature. The term polypeptide or protein as used herein encompasses any amino acid sequence and includes, but may not be limited to, modified sequences such as glycoproteins. The term polypeptide is specifically intended to cover naturally occurring proteins, as well as those that are recombinantly or synthetically produced.

Substantially purified polypeptide as used herein refers to a polypeptide that is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative amino acid substitution tables providing functionally similar amino acids are well known to one of ordinary skill in the art. The following six groups are examples of amino acids that are considered to be conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

A non-conservative amino acid substitution can result from changes in: (a) the structure of the amino acid backbone in the area of the substitution; (b) the charge or hydrophobicity of the amino acid; or (c) the bulk of an amino acid side chain. Substitutions generally expected to produce the greatest changes in protein properties are those in which: (a) a hydrophilic residue is substituted for (or by) a hydrophobic residue; (b) a proline is substituted for (or by) any other residue; (c) a residue having a bulky side chain, e.g., phenylalanine, is substituted for (or by) one not having a side chain, e.g., glycine; or (d) a residue having an electropositive side chain, e.g., lysyl, arginyl, or histadyl, is substituted for (or by) an electronegative residue, e.g., glutamyl or aspartyl.

Variant amino acid sequences may, for example, be 80%, 90% or even 95% or 98% identical to the native amino acid sequence. Programs and algorithms for determining percentage identity can be found at the NCBI website.

Primary tumor: The original tumor. A tumor located at the original tumor site, as opposed to a metastatic or secondary tumor, which is located at a site distal to the primary tumor.

Protein: A biological molecule expressed by an encoding nucleic acid molecule (for example, a gene) and comprised of amino acids. Proteins are a subset of the broader molecular class “peptide.”

Purified: The term purified does not require absolute purity; rather, it is intended as a relative term. Thus, for example, a “purified” protein preparation is one in which the protein is more enriched than the protein is in its generative environment, for instance within a cell or in a biochemical reaction chamber. Preferably, a preparation of protein is purified such that the protein represents at least 50%, at least 60%, at least 70%, at least 75%, at least 80%, at least 85%, at least 90%, at least 95%, or at least 99% of the total protein content of the preparation.

Recombinant nucleotide: A recombinant nucleotide is one that has a sequence that is not naturally occurring or has a sequence that is made by an artificial combination of two otherwise separated segments of nucleotide sequence. This artificial combination can be accomplished by chemical synthesis or, more commonly, by the artificial manipulation of isolated segments of nucleic acids, for example, by genetic engineering techniques. Similarly, a recombinant protein is one encoded for by a recombinant nucleotide.

Sequence identity: The similarity between two nucleic acid sequences, or two amino acid sequences, is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman (Adv. Appl. Math. 2: 482, 1981); Needleman and Wunsch (J. Mol. Biol. 48: 443, 1970); Pearson and Lipman (PNAS USA 85: 2444, 1988); Higgins and Sharp (Gene, 73: 237-244, 1988); Higgins and Sharp (CABIOS 5: 151-153, 1989); Corpet et al. (Nuc. Acids Res. 16: 10881-10890, 1988); Huang et al. (Comp. Appls Biosci. 8: 155-165, 1992); and Pearson et al. (Meth. Mol. Biol. 24: 307-31, 1994). Altschul et al. (Nature Genet., 6: 119-129, 1994) presents a detailed consideration of sequence alignment methods and homology calculations.

The alignment tools ALIGN (Myers and Miller, CABIOS 4:11-17, 1989) or LFASTA (Pearson and Lipman, 1988) may be used to perform sequence comparisons (Internet Program© 1996, W. R. Pearson and the University of Virginia, “fasta20u63” version 2.0u63, release date December 1996). ALIGN compares entire sequences against one another, while LFASTA compares regions of local similarity. These alignment tools and their respective tutorials are available on the Internet at the NCSA Website. Alternatively, for comparisons of amino acid sequences of greater than about 30 amino acids, the “Blast 2 sequences” function can be employed using the default BLOSUM62 matrix set to default parameters, (gap existence cost of 11, and a per residue gap cost of 1). When aligning short peptides (fewer than around 30 amino acids), the alignment should be performed using the “Blast 2 sequences” function, employing the PAM30 matrix set to default parameters (open gap 9, extension gap 1 penalties). The BLAST sequence comparison system is available, for instance, from the NCBI web site; see also Altschul et al., J Mol. Biol. 215:403-410, 1990; Gish. & States, Nature Genet. 3:266-272, 1993; Madden et al. Meth. Enzymol. 266:131-141, 1996; Altschul et al., Nucleic Acids Res. 25:3389-3402, 1997; and Zhang & Madden, Genome Res. 7:649-656, 1997.

Orthologs (equivalent to proteins of other species) of proteins are in some instances characterized by possession of greater than 75% sequence identity counted over the full-length alignment with the amino acid sequence of specific protein using ALIGN set to default parameters. Proteins with even greater similarity to a reference sequence will show increasing percentage identities when assessed by this method, such as at least 80%, at least 85%, at least 90%, at least 92%, at least 95%, or at least 98% sequence identity. In addition, sequence identity can be compared over the full length of one or both binding domains of the disclosed fusion proteins.

When significantly less than the entire sequence is being compared for sequence identity, homologous sequences will typically possess at least 80% sequence identity over short windows of 10-20, and may possess sequence identities of at least 85%, at least 90%, at least 95%, or at least 99% depending on their similarity to the reference sequence. Sequence identity over such short windows can be determined using LFASTA; methods are described at the NCSA Website. One of skill in the art will appreciate that these sequence identity ranges are provided for guidance only; it is entirely possible that strongly significant homologs could be obtained that fall outside of the ranges provided. Similar homology concepts apply for nucleic acids as are described for protein.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found in Sambrook et al. (In Molecular Cloning: A Laboratory Manual, Cold Spring Harbor, N.Y., 1989) and Tijssen (Laboratory Techniques in Biochemistry and Molecular Biology Part I, Ch. 2, Elsevier, N.Y., 1993).

Nucleic acid sequences that do not show a high degree of identity may nevertheless encode similar amino acid sequences, due to the degeneracy of the genetic code. It is understood that changes in nucleic acid sequence can be made using this degeneracy to produce multiple nucleic acid sequences that each encode substantially the same protein.

Specific binding agent: An agent that binds substantially only to a defined target. Thus a peptide-specific binding agent binds substantially only the defined peptide, or a peptide region within a protein, such as a fusion protein. As used herein, the term “[X] specific binding agent,” where [X] refers to a specific protein or peptide, includes anti-[X] antibodies (and functional fragments thereof) and other agents (such as soluble receptors) that bind substantially only to [X]. It is contemplated that [X] can be a family of closely-related proteins (for instance, closely-related TGF-βs) that are recognized by one specific binding agent. An antibody is one example of a specific binding agent.

Subject: Living multi-cellular vertebrate organisms, a category that includes both human and non-human mammals.

TGF-β family of proteins: A family of secreted signaling molecules involved in a number of cellular and developmental processes in eukaryotic cells, including inflammation, immune surveillance, and neoplasia. Members of the TGF-β family of proteins include, but are not limited to: TGF-β2, TGF-β3, TGF-β1, TGF-β4 (chicken), TGF-β5 (Xenopus), GDF-9 (mouse/human), BMP-16/nodal (mouse), Fugacin (Xenopus), BMP3, Sumitomo-BIP/GDF-10 (mouse), ADMP (Xenopus), BMP-9, Dorsalin-1 (Chicken), BMP-10, BMP-13/GDF-6 (mouse), Radar (Zebrafish), GDF-1/CDMP-1 (mouse/human), BMP-12/GDF-7 (mouse), BMP-5, BMP-6, BMP-7/OP-1, BMP-8/OP-2, PC8/OP-3 (mouse), 60A (Drosophila), BMP-2, BMP-4, Decapentaplegic (Drosophila), Vg-1 (Xenopus), Univin (sea urchin), Vgr-2/GDF-3, GDF-1, Screw (Drosophila), BMP-11, GDF-8, ActivinβC, ActivinβD (Xenopus), ActivinβE, BMP-14/GDF-12, ActivinβA, ActivinβB, GDF-14, Mullerian inhibiting substance, and α-inhibin. The term “TGF-β” is used generally herein to mean any isoform of TGF-β, provided the isoform has immunosuppressive activity. Methods are disclosed herein of using agents to block the immunosuppressive effects of TGF-β.

The term TGF-β family protein function includes all functions or activities that are associated with a TGF-β family protein, including for instance secondary folding of each TGF-β monomer, tertiary association between the members of the multimeric (for example, homodimeric) TGF-β complex, maturation by cleavage and/or removal of the pro-region (LAP), secretion of the protein from the cell in which it was translated, specific receptor binding, and down-stream activities that result from the binding of a TGF-β family ligand protein with its cognate receptor(s). Such downstream activities include (depending on the TGF-β family member examined and the system used), for instance, regulation of cell growth (proliferation), stimulation of cell growth or proliferation, stimulation of cell differentiation, inhibition of cell growth or proliferation, regulation of cytokine production, induction of cellular differentiation, cell cycle inhibition, control of adhesion molecule expression, stimulation of angiogenesis, induction of leukocyte chemotaxis, induction of apoptosis, suppression of lymphocyte activation, suppression of inflammation, enhancement of wound healing by mechanisms including, stimulation of synthesis of matrix proteins, regulation of immunoglobulin production, including isotype switch recombination, and suppression of tumorigenesis.

Different members of the TGF-β family have different biological specificities and activities. Specificities of the listed TGF-β family proteins are known to one of ordinary skill in the art. See, for instance, Doetschman, Lab. Anim. Sci. 49:137-143, 1999; Letterio and Roberts, Annu. Rev. Immunol. 16:137-61:137-161, 1998; Wahl, J. Exp. Med. 180:1587-1590, 1994; Letterio and Roberts, J. Leukoc. Biol. 59:769-774, 1996; Piek et al., FASEB J. 13:2105-2124, 1999; Heldin et al., Nature 390:465-471, 1979; and De Caestecker et al., J. Nat'l Cancer Inst., 92:1388-1402, 2000.

TGF-β mutants, including fragments of TGF-β and TGF-β peptides, that retain the ability to bind a TGF-β receptor but cannot induce a TGF-β signaling pathway are encompassed by the disclosure. Also encompassed by the disclosure are TGF-β point mutants that retain the ability to bind a TGF-β receptor but cannot induce the TGF-β signaling pathway. Certain TGF-β mutants, such as those disclosed herein, are “neutralizing” molecules.

TGF-β signaling pathway: TGF-β transmits a signal across a cell membrane by stimulating the formation of specific heteromeric complexes of type I and type II serine/threonine kinase receptors (for example, a TGF-β receptor). The type II receptors bind ligand (for example, a TGF-β), and phosphorylate and activate the type I receptors, whereas the type I receptors are responsible for the specificity of downstream signaling. The downstream intracellular molecules, or effectors, of the phosphorylated type I receptor are known as Smads.

Smads, the only substrates for type I receptor kinases known to have a signaling function, have two conserved domains, the N-terminal Mad homology 1 and the C-terminal Mad homology 2 domains. Smads are ubiquitously expressed throughout development and in all adult tissues. Functionally, Smads fall into three subfamilies: receptor-activated Smads (R-Smads; Smad1, Smad2, Smad3, Smad5, Smad8), which become activated by type I receptors; common mediator Smads (Co-Smads; Smad4), which oligomerize with activated R-Smads; and inhibitory Smads (I-Smads; Smad 6 and Smad7), which are induced by TGF-β family members.

Activated TGF-β receptors phosphorylate Smad2 and Smad3. Phosphorylation of the C-terminal serine residues in R-Smads by type I receptor kinases is a crucial step in TGF-β signaling. The two most C-terminal serine residues become phosphorylated and, together with a third non-phosphorylated serine residue, form an evolutionarily conserved SSXS motif in all R-Smads. Unphosphorylated Smad proteins exist primarily as monomers, and upon phosphorylation, R-Smads form homo-oligomers, which quickly convert to hetero-oligomers containing the Co-Smad, Smad4.

All R-Smads, mammalian Smad4, and Xenopus Smad4α reside in the cytoplasm. However, heteromeric R-Smad/Co-Smad complexes are found in the nucleus, thus the Smads must translocate to the nucleus. The NH1 domains of all eight Smads each contain a lysine-rich motif that, in the case of Smad1 and Smad3, has been shown to function as a nuclear localization signal.

All Smads have transcriptional activity. Heteromeric R-Smad/Co-Smad complexes are the transcriptionally relevant entities in vivo. Smad3 and Smad4 bind directly, but with low affinity to Smad binding elements (SBEs), through a conserved P-hairpin loop in the MH1 domain. Additional MH1 sequences, such as α-helix 2, contribute to SBE DNA-binding by Smad3. Because of the low affinity to SBEs, DNA-binding co-factors must be involved in providing a tight and highly specific recognition of the regulatory elements in target genes. The choice of target gene by an activated Smad complex is made by the association of this complex with specific DNA-binding co-factors. Examples of such co-factors include FAST, OAZ, AP-1, TFE3, and AML proteins. Once a Smad complex binds DNA it may control the transcription of target genes, for example by altering nucleosome structure (Massague and Chen, Genes and Development 14:627-644, 2000; Moustakas et al., J Cell Sci. 114:4359-4369, 2001).

Agents, as disclosed herein, that bind TGF-β, the TGF-β receptor, or any of the TGF-β receptor's downstream signaling partners can block the TGF-β signaling pathway (a blockade of TGF-β signaling). In one embodiment, the agent is a neutralizing agent that results in an inhibition of the activity of the molecule to which it binds. TGF-β mutants, including fragments of TGF-β and TGF-β peptides, which retain the ability to bind a TGF-β receptor but cannot induce the TGF-β signaling pathway are encompassed by the disclosure. Also encompassed by the disclosure are TGF-β point mutants that retain the ability to bind a TGF-β receptor but cannot induce the TGF-β signaling pathway. A blockade of TGF-β signaling can prevent, for example, the phosphorylation of a type I receptor, the phosphorylation of a Smad, the binding of a Smad to a Smad binding element, or the transcription of a target gene.

Therapeutically effective amount: A quantity sufficient to achieve a desired effect in a subject being treated. For instance, when referring to the combination including an anti-TGF-β antibody and a tumor peptide antigen, or an anti-TGF-β antibody and an irradiated whole cell, this can be the amount necessary to induce a dose-dependent effect. Examples of dose-dependent effects include:

(i) the amount of neutralizing anti-TGF-β antibody and tumor peptide antigen that, when administered to a subject in combination, can both inhibit an immunosuppressive effect of TGF-β and induce (or enhance) an immune response resulting in a synergistic inhibition of tumor growth, compared to the anti-TGF-β antibody or the tumor peptide alone; and

(ii) the amount of neutralizing anti-TGF-β antibody and irradiated whole cell that, when administered prophylactically to a subject in combination, can both inhibit an immunosuppressive effect of TGF-β and enhance an immune response resulting in a synergistic inhibition of tumor growth, compared to the anti-TGF-β antibody or the irradiated whole cell alone.

An effective amount of an agent may be administered in a single dose, or in several doses, for example daily, during a course of treatment. However, the effective amount of agent will be dependent on the agent applied, the subject being treated, the severity and type of the affliction, and the manner of administration of the agent. For example, a therapeutically effective amount of the neutralizing anti-TGF-β antibody 1D11.16 can vary from about 0.01 mg/kg body weight to about 1 g/kg body weight. In one specific, non-limiting example, a therapeutically effective amount of the neutralizing anti-TGF-β antibody 1D11.16 is about 3-4 mg/kg body weight. In another specific, non-limiting example, a therapeutically effective amount of the E7₍₄₉₋₅₇₎ peptide is 100 μg per dose. In other specific, non-limiting examples, a therapeutically effective amount of the irradiated CT26 cells is between about 1×10² cells and about 1×10⁸ cells per dose. In yet other specific, non-limiting examples, a therapeutically effective amount of the irradiated CT26 cells is between about 1×10⁴ cells and about 1×10⁶ cells per dose. In a further specific, non-limiting example, a therapeutically effective amount of the irradiated CT26 cells is 1×10⁵ cells per dose.

The agents disclosed herein have equal application in medical and veterinary settings. Therefore, the general term “subject being treated” is understood to include all animals (for example, humans, apes, dogs, cats, horses, and cows).

Treatment: Refers to both prophylactic inhibition of disease (such as tumor recurrence or metastasis) and therapeutic interventions to alter the natural course of an untreated disease process, such as tumor growth. Treatment of a tumor includes, for instance, the surgical removal of the tumor. Treatment of a tumor can also include chemotherapy, immunotherapy, or radiation therapy. Two or more methods of treating a tumor can be provided to a subject in combination. Treatment of a subject, as the term is used herein, includes preventing further growth of an existing tumor, enhancing tumor regression, inhibiting tumor recurrence, or inhibiting tumor metastasis.

Tumor: A neoplasm that may be either malignant or non-malignant (benign). Tumors of the same tissue type are tumors originating in a particular organ (such as breast, prostate, bladder or lung). Tumors of the same tissue type may be divided into tumor of different sub-types (a classic example being bronchogenic carcinomas (lung tumors) which can be an adenocarcinoma, small cell, squamous cell, or large cell tumor). Breast cancers can be divided histologically into scirrhous, infiltrative, papillary, ductal, medullary and lobular. Unless it is clear from the context, it is intended that the term tumor includes reference to non-solid tumors, which may more generally be called neoplasms, and particularly malignant neoplasms such as leukemias.

Tumor recurrence: The return of a tumor, at the same site as the original (primary) tumor, after the tumor has been removed surgically, by drug or other treatment, or has otherwise disappeared. Tumor recurrence often occurs even though a tumor appears to be completely eradicated (by any method) or has disappeared. However, the eradication is often not complete and, as an established blood supply exists, a tumor can recur. A subject that has had a tumor removed by any method (for example, surgical removal, drug or other treatment) or that has had a tumor disappear, is at risk for recurrence of a tumor.

Unless otherwise explained, all technical and scientific terms used herein have the same meaning as commonly understood by one of ordinary skill in the art to which this invention belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Description of Several Specific Embodiments

The disclosure provides in a first embodiment a method of enhancing tumor regression in a subject (for instance, a human subject), which method involves administering to the subject a combination including a therapeutically effective amount of an antibody, wherein the antibody inhibits a TGF-β in the subject, and an immunogenic agent, wherein the agent is a tumor peptide, wherein the subject has a tumor or is at risk of developing a tumor, thereby enhancing tumor regression in the subject.

The disclosure provides in a second embodiment a method of enhancing tumor regression in a subject (for instance, a human subject), which method involves administering to the subject a combination including a therapeutically effective amount of an antibody, wherein the antibody inhibits a TGF-β in the subject, and an immunogenic agent, wherein the agent is inactivated whole cells, wherein the subject has a tumor or is at risk of developing a tumor, thereby enhancing tumor regression in the subject.

In specific examples of such methods of enhancing tumor regression in a subject, the antibody in the combination is either a polyclonal antibody or a monoclonal antibody. In one specific, non-limiting example, the monoclonal antibody is specific for TGF-β, such as the monoclonal antibody obtained from hybridoma 1D11.16 (ATCC Accession No. HB 9849). In other examples, the monoclonal antibody is a human monoclonal antibody specific for TGF-β, such as for instance GC1008 (Genzyme Corp., Cambridge, Mass.). For instance, in some examples, the anti-TGF-β antibody inhibits TGF-β from binding a TGF-β receptor, thereby blocking an immunosuppressive effect in the subject. In other examples, inhibiting TGF-β increases immunosurveillance by lymphocytes in the subject.

In specific examples of such methods of enhancing tumor regression in a subject, the immunogenic tumor peptide in the combination is a Human Papilloma Virus (HPV)-16 peptide, such as an E6 or an E7 peptide. In one specific non-limiting example, the E7 peptide is the E7₍₄₉₋₅₇₎ peptide epitope. In other examples of the methods, the immunogenic inactivated whole cells are irradiated cells. In one specific non-limiting example, the irradiated whole cells are irradiated CT26 murine colorectal tumor cells.

The tumor referred to in the methods provided herein may be a benign tumor, a malignant tumor, a primary tumor, or a metastasis. The tumor can include a carcinoma, a sarcoma, a leukemia, or a tumor of the nervous system. In other examples, the tumor includes a breast tumor, a liver tumor, a pancreatic tumor, a gastrointestinal tumor, a colon tumor a uterine tumor, a ovarian tumor, a cervical tumor, a testicular tumor, a brain tumor, a skin tumor, a melanoma, a retinal tumor, a lung tumor, a kidney tumor, a bone tumor, a prostate tumor, a nasopharyngeal tumor, a thyroid tumor, a leukemia, or a lymphoma.

The combination of agents used in the methods can be administered, for instance, intravenously, subcutaneously, intradermally, or intramuscularly. In specific examples, the combination of agents is administered prior to detection of the tumor or following detection of the tumor.

### IV. Method of Affecting Tumor Growth by Blocking the TGF-β Signaling Pathway and Administering an Immunogenic Agent

Methods are disclosed herein of enhancing an anti-tumor immunity in a subject by administering a combination of agents, wherein the combination of agents produces a synergistic response that affects tumor growth, for example preventing further growth of an existing tumor, enhancing tumor regression, inhibiting tumor recurrence, or inhibiting tumor metastasis. The combination of agents includes a first agent that blocks the TGF-β signaling pathway, thereby blocking TGF-β's immunosuppressive effects. The combination also includes a second agent, such as an immunogenic agent (for example a tumor peptide antigen), that generates an immune response. The disclosed method of administering the two (or more) agents to a subject is more effective than the administration of each agent individually, or the sum of their individual effects. Although the agents may be administered in this order, administration of the combination of agents is not bound to this order.

The disclosed methods synergistically prevent or inhibit the growth of a tumor or enhance the regression of a tumor, for instance by any measure amount. The term “inhibit” does not require absolute inhibition. Similarly, the term “prevent” does not require absolute prevention. Inhibiting the growth of a tumor or enhancing the regression of a tumor includes reducing the size of an existing tumor. Preventing the growth of a tumor includes preventing the development of a primary tumor or preventing further growth of an existing tumor. Reducing the size of a tumor includes reducing the size of a tumor by a measurable amount, for example at least 5%, at least 10%, at least 20%, at least 30%, at least 40%, at least 50%, at least 60%, at least 70%, at least 80%, at least 85%, at least 90%, at least 95%, at least 99%, or 100%.

### Blocking the TGF-β Signaling Pathway

The mechanism of down-regulation of tumor immunosurveillance by CTLs, caused by the immunosuppressive effects of TGF-β on CTLs, has been studied using a mouse tumor model in which tumors show a growth-regression-recurrence pattern after tumor inoculation (Matsui et al., J. Immunol. 163:184, 1999). With this mouse tumor model, it was demonstrated that tumor recurrence was the result of incomplete elimination of tumor cells by CTLs that were negatively regulated by IL-13 produced by CD4⁺ CD1d-restricted NKT cells through the IL-4Roc-STAT6 signaling pathway (Terabe et al., Nature Immunol. 1:515, 2000). It has also been demonstrated that IL-13 made by these CD4⁺ CD1d-restricted NKT cells induces CD11b⁺Gr-1⁺ non-lymphoid cells of myeloid origin to produce TGF-β (Terabe et al., J Exp Med. 198(11): 1741-52, 2003). It is also known that TGF-β causes the down-regulation of CD8⁺ CTL-mediated tumor immunosurveillance.

Thus, methods are disclosed herein of affecting tumor growth in a subject (for example preventing further growth of an existing tumor, enhancing tumor regression, inhibiting tumor recurrence, or inhibiting tumor metastasis) by administering a combination of agents, wherein one of the agents in the combination blocks TGF-β's immunosuppressive effects. Examples of the methods include administering to a subject a therapeutically effective amount of an agent which, for example, directly or indirectly blocks TGF-β binding to the TGF-β receptor, thereby blocking the TGF-β signaling pathway. In alternative examples, the agent blocks a different step in the TGF-β signaling pathway, for instance, downstream of TGF-β binding to a receptor. Administration of an agent which blocks the TGF-β signaling pathway is particularly effective against tumors that have escaped CTL immunosurveillance as a result of the immunosuppressive effects of TGF-β. Thus, blocking the TGF-β signaling pathway affects tumors in a subject by preventing further growth of an existing tumor, enhancing tumor regression, inhibiting tumor recurrence, or inhibiting tumor metastasis.

A subset of T cells, CD4⁺CD25⁺ cells, has been shown to down regulate many immune responses including auto-immune responses and anti-tumor immune responses which are mediated by T cells (Sakaguchi et al. J. Immunol. 155:115, 1995). One of the suggested mechanisms of the CD4⁺CD25⁺ cells is through TGF-β expressed on their surface (Nakamura et al., J. Exp. Med. 194:629, 2001). It has also been shown that TGF-β plays a critical role in induction of this immunosuppressive T cell population (Fantini et al., J Immunol 172:5149, 2004). Therefore, blockade of TGF-β and its signaling pathway may also enhance T cell immune responses to tumors by blocking development and effector function of CD4⁺CD25⁺ T cells.

### Enhancing an Activity of an Immune Cell or an Immune Response in a Subject by Blocking the TGF-β Signaling Pathway

The disclosure provides methods of enhancing the activity of an immune cell by administering a combination of agents, wherein one of the agents in the combination blocks the TGF-β signaling pathway, thereby affecting tumor growth in a subject. Immune cells that are susceptible to a block in the TGF-β signaling pathway are those cells that express the TGF-β receptor.

Immune cells include leukocytes (for instance, neutrophils, eosinophils, monocytes, basophils, macrophages, B cells, T cells, dendritic cells, and mast cells), as well as other types of cells involved in an immune response. Methods provided herein include contacting an immune cell that expresses a TGF-β receptor with an agent that blocks the TGF-β signaling pathway. In one embodiment, the immune cell is a lymphocyte, such as a T cell or a B cell. In other embodiments, the immune cell is a CTL, a CD8⁺ CTL, a CD4⁺ T cell, a γδ TCR+T cell (which has been shown to play some role in anti-tumor protective immunity; see, e.g., Girardi et al, Science 294:605, 2001), an NK cell, or an NKT cell. In a further embodiment, the immune cell is a granulocyte. The immune cell can be either in vivo or in vitro. The agent can either bind TGF-β, a TGF-β receptor, or a TGF-β receptor downstream signaling molecule.

In one embodiment, the activity of an immune cell is enhanced in a subject, following the administration of a combination of agents, wherein one of the agents blocks the TGF-β signaling pathway. Immune cells having an enhanced activity, for example increased tumor immunosurveillance, following the administration of the agent include cells that express a TGF-β receptor, such as a CTL. In one embodiment, the immune cell with the enhanced activity is in a subject suffering from a tumor that has escaped CTL immunosurveillance. In another embodiment, an enhanced activity of an immune cell, such as enhanced CTL immunosurveillance, enhances anti-tumor immunity in a subject and prevents further growth of an existing tumor, enhances tumor regression, inhibits tumor recurrence, or inhibits tumor metastasis.

The disclosure also provides methods of enhancing an immune response in a subject by administering a combination of agents, wherein one of the agents blocks the TGF-β signaling pathway. In one embodiment, an enhanced immune response, for example increased tumor immunosurveillance, enhances the anti-tumor immunity of a subject, thereby affecting tumor growth.

The disclosed method includes administering to the subject a therapeutically effective amount of an agent, which blocks the TGF-β signaling pathway, to enhance the immune response. In one embodiment, the immune response is a T cell response. In another embodiment, the immune response involves a TGF-β receptor-expressing cell. The cell expressing a TGF-β receptor can be, but is not limited to, a CTL, a CD8⁺ CTL, a CD4⁺ T cell, a CD4⁺ CD 1d-restricted T cell, an NK cell, or an NKT cell. In a further embodiment, the immune response is CTL-mediated immunosurveillance. In one embodiment, a subject with an enhanced immune response is suffering from a tumor that has escaped CTL immunosurveillance. In another embodiment, an enhanced immune response prevents further growth of an existing tumor, enhances tumor regression, inhibits tumor recurrence, or inhibits tumor metastasis in a subject.

A method is also disclosed herein for enhancing a T cell-mediated immune response. The method includes administering to the subject a therapeutically effective amount of an agent, which blocks the TGF-β signaling pathway, to improve a T cell-mediated immune response. In one embodiment, the T cell-mediated immune response is CTL-mediated immunosurveillance. In another embodiment, the T cell-mediated immune response is an NKT cell response. In a further embodiment, T cell-mediated immune response is a CD4+CD1d-restricted T cell response.

Agents that Block the TGF-β Signaling Pathway

Agents that block the TGF-β signaling pathway, including neutralizing agents, block the immunosuppressive effects of TGF-β and enhance an activity of an immune cell, such as CTL immunosurveillance, or an immune response in a subject, thereby enhancing anti-tumor immunity in a subject. In one embodiment, an agent affects tumor growth. In another embodiment, an agent inhibits the recurrence of a tumor that has escaped CTL immunosurveillance. In other embodiments, an agent affects tumors by preventing further growth of an existing tumor, enhancing tumor regression, inhibiting tumor recurrence, or inhibiting tumor metastasis in a subject. The agent is intended to be used with a second agent, for example an immunogenic agent, and can be used with a third agent, a fourth agent, or additional agents.

The agent that blocks the TGF-β signaling pathway can be any substance, including, but not limited to, an antagonist, an antibody, a chemical compound, a small molecule, a peptide mimetic, a peptide, or a polypeptide. The agent is preferably a non-toxic agent. An agent that blocks the TGF-β signaling pathway can be, for example, an enzyme (for example, a kinase or a phosphorylase) or another catalytic molecule that selectively binds and alters the function and/or the activity of a protein in the TGF-β signaling pathway. For example, proteins can be functional when phosphorylated and nonfunctional when de-phosphorylated. A functional, phosphorylated, protein can become nonfunctional when exposed to a de-phosphorylating agent such as a phosphorylase. Thus, a cell that is active as the result of expressing a functional protein, can become inactivated when it is in contact with an agent that inhibits (neutralizes) the function of the protein. The reverse is also true. For example, a cell that is inactive as the result of expressing a functional protein, can become activated when it is in contact with an agent that inhibits (neutralizes) the function of the protein.

In one embodiment, the agent that blocks the TGF-β signaling pathway is a neutralizing agent. An agent can neutralize (inhibit an activity of) a molecule in the TGF-β signaling pathway by specifically binding it, thereby preventing the molecule from performing at least one function in the pathway. For example, a neutralizing agent can prevent a molecule in the pathway from interacting with other molecules. In one specific, non-limiting example, a neutralizing agent prevents TGF-β from specifically binding the TGF-β receptor.

In one embodiment, the agent that blocks the TGF-β signaling pathway is an antagonist. An antagonist is any substance that tends to nullify, or neutralize, the action of a molecule in the TGF-β signaling pathway, for example a drug that binds to a receptor, such as a TGF-β receptor, without eliciting a biological response. In one embodiment, the antagonist is a chemical compound that neutralizes TGF-β directly. In other embodiments, the antagonist is a chemical compound that neutralizes the TGF-β receptor or at least one of its downstream signaling molecules (for example, Smad 2, Smad3, or Smad 4), or a Smad complex DNA-binding co-factor.

In one embodiment, the agent that blocks the TGF-β signaling pathway interacts (for example, specifically binds) with the TGF-β molecule directly. The agent in some embodiments is an anti-TGF-β antibody. Such an anti-TGF-β antibody can be a polyclonal antibody or a monoclonal antibody. In one specific, non-limiting example, the anti-TGF-β antibody is a monoclonal antibody obtained from the hybridoma 1D11.16 (ATCC Accession No. HB 9849) binds TGF-β. In another non-limiting example, the monoclonal antibody is a human monoclonal antibody specific for TGF-β, such as for instance GC1008 (Genzyme Corp., Cambridge, Mass.). Agents, such as the 1D11.16 or GC1008 antibody, can bind TGF-β and neutralize its activity by preventing it from binding TGF-β receptor(s).

Alternatively, an agent that blocks the TGF-β signaling pathway can form a complex with a ligand, such as TGF-β so that it is still capable of binding a receptor, such as a TGF-β receptor, but the ligand:agent complex is incapable of activating the receptor and transmitting a signal.

An agent that blocks the TGF-β signaling pathway can specifically bind a receptor, such as the TGF-β receptor, and prevent the receptor from transmitting a signal across the cell membrane into the cell. More specifically, an agent can specifically bind a receptor, such as the TGF-β receptor, at its ligand-binding site thereby preventing a ligand, such as TGF-β from binding to the receptor. As disclosed herein, TGF-β is used generally herein to mean any isoform of TGF-β, provided the isoform has immunosuppressive activity. In one specific, non-limiting example, the agent is an anti-TGF-β receptor antibody. In another specific, non-limiting example, the agent is a TGF-β mutant. TGF-β mutants include fragments of TGF-β and TGF-β peptides that retain the ability to bind a TGF-β receptor but cannot induce the TGF-β signaling pathway. TGF-β mutants also include TGF-β point mutants that retain the ability to bind a TGF-β receptor but cannot induce the TGF-β signaling pathway, or induce it only at a low level compared to the wildtype TGF-β.

An agent that blocks the TGF-β signaling pathway can also specifically bind one or more of the TGF-β receptor's downstream signaling molecules. For example, some agents neutralize TGF-β activity by specifically binding a downstream signaling molecule and preventing the transmission of an intracellular TGF-β signal. TGF-β downstream signaling molecules include, but are not limited to, Smad2, Smad3, Smad4, or Smad complex DNA-binding co-factors.

In one specific, non-limiting, embodiment, a neutralizing agent that blocks the TGF-β signaling pathway is a soluble TGF-β receptor. The soluble TGF-β receptor specifically binds TGF-β and competes with the TGF-β cell surface receptor for any available TGF-β. Preventing TGF-β from binding its endogenous receptor neutralizes the activity of TGF-β, provided that sufficient soluble TGF-β receptor is present in order to bind all of the available TGF-β ligand.

The TGF-β receptor can be expressed in a lymphocyte, such as a T lymphocyte. More specifically, the TGF-β receptor can be expressed in a CTL. Thus, the method of using an agent to neutralize the activity of TGF-β prevents TGF-β signaling in a TGF-β receptor-expressing CTL.

### Tumor Polypeptides and Peptides as Immunogenic Agents

The current disclosure provides methods of using combinations of agents to affect tumor growth, wherein one of the agents is an immunogenic agent, such as a antigenic portions of a cell (for example polypeptides, peptides, membranes, etc.). In one embodiment, the immunogenic agent induces an immunogenic response in a subject. The immunogenic agent may be any immunogenic polypeptide, for example a polypeptide expressed by a tumor cell (a tumor antigen). In one embodiment, the polypeptides and peptides are obtained from a subject's tumor cells. In another embodiment, the polypeptides and peptides are obtained from lysed tumor cells from that subject. The polypeptide may be a full-length polypeptide, or a polypeptide that has been enzymatically processed in vitro or in vivo into smaller polypeptides or peptides. Alternatively, the polypeptides and peptides may be chemically synthesized using well known methods of polypeptide/peptide synthesis. The immunogenic agent is intended to be used with a second agent, and can be used with a third agent, a fourth agent, or additional agents, for example with an agent that blocks the TGF-β signaling pathway.

Immunogenic polypeptides may be any length. For example, the polypeptides may be 25, 30, 50, 100, 200, 300, or more amino acids in length. Specific, non-limiting examples of an immunogenic polypeptide include human papilloma virus 16 E6 and E7 proteins. In one embodiment, peptides used as immunogenic agents are linear polymers of approximately 6-24 amino acids in length. In other embodiments, peptides used as immunogenic agents are linear polymers of approximately 8-20, 10-16, or 12-14 amino acids in length. In one specific, non-limiting example, peptides used as an immunogenic agent are linear polymers of nine amino acids. One specific, non-limiting example of a nine amino acid long peptide is the E7₍₄₉₋₅₇₎ peptide (SEQ ID NO: 1).

Another specific, non-limiting example of a nine amino acid long peptide is AH1 peptide (SPSYVYHQF; SEQ ID NO: 3). The AH1 peptide is a CTL epitope of gp70 expressed in the CT26 tumor cell line. Yet other contemplated antigenic peptides are derived from gp 100, a melanoma-specific antigen which is unrelated to CT26. By way of non-limiting example, one gp100-derived human CTL epitope presented by HLA-A2 (gp100₂₀₉₋₂₁₇, ITQVPFSV; SEQ ID NO: 4) is specifically contemplated as a peptide useful in combination with a blockade of a TGF-β signaling pathway to treat, for instance, melanoma patients. Also contemplated for use in combined agent treatment methods are peptides derived from TCR-γ alternate reading frame protein (TARP), such as for instance SEQ ID NO: 5 (FLRNFSLML) and SEQ ID NO: 6 (FVFLRNFSL), for use in treatment of, for instance, breast or prostate cancer patients. For a discussion of TARP and its antigenicity, see Wolfgang et al., Cancer Res. 61:8122-8126, 2001; Oh et al., Cancer Res. 64:2610-2618, 2004; and Carlsson et al., Prostate 61:161-170, 2004.

Cyclic peptides, branched peptides, peptomers (cross-linked peptide polymers) and other complex multimeric structures, as well as peptides conjugated to other molecules, which mimic conformational structures of peptides found in nature, are encompassed by this disclosure.

The immunogenic polypeptides and peptides may include CTL-stimulatory epitopes, T-helper cell stimulatory epitopes, B-cell stimulatory epitopes, or combinations of two or more such types of epitopes. One aspect of embodiments provided herein is that the immunogenic polypeptide and peptide sequences each contain one or more antibody-binding or class I or class II MHC-binding epitopes. Included epitopes also may be B-cell epitopes, which elicit antibody-mediated immune responses upon binding to antibody receptors on the surface of a B-cell. The immunogenic polypeptides and peptides also include those epitopes that may be immunodominant and that induce specific immune functions.

Optionally, immunogenic polypeptides and peptides are covalently linked to larger molecules (carriers), thereby enhancing immunogenicity of the polypeptide or peptide. In one embodiment, the carriers contain T helper epitopes (preferably strong versus weak epitopes). Examples of carrier proteins include tetanus toxoid, Pseudomonas aeruginosa toxin A, beta-galactosidase, Brucella abortus, keyhole limpet hemocyanin, influenza virus hemagglutinin, influenza virus nucleoprotein, hepatitis B core antigens, and hepatitis B surface antigens. In one embodiment, the carriers provide T cell help or facilitate the presentation of the polypeptide or peptide. The immunogenicity of polypeptides and peptides can be further enhanced by covalent linkage with plasma α-2 macroglobulin, β-2 microglobulin, or light and heavy immunoglobulin chains. Direct covalent linkage, or cross-linking, is performed using well known methods.

Covalent fusion of polypeptides and peptides to lipids may also enhance immunogenicity. In one embodiment, polypeptides or peptides covalently fused to a lipid produces a more efficient induction of CTLs.

### Inactivated Whole Cells as Immunogenic Agents

The current disclosure provides methods of using combinations of agents to affect tumor growth, wherein one of the agents is an immunogenic agent, such as inactivated whole cells. In one embodiment, the immunogenic agent induces an immunogenic response in a subject. Immunogenic whole cells include cells that are treated in such a way that they can no longer cause disease. In one embodiment, the cell is killed but still retains its immunogenicity. The immunogenic agent is intended to be used with a second agent, and can be used with a third agent, a fourth agent, or additional agents, for example with an agent that blocks the TGF-β signaling pathway.

Immunogenic whole cells can be derived from a subject's tumor, for example from biopsy tissue, from explants of a removed tumor, or from cell culture of the subject's tumor cells. One specific, non-limiting example of a tumor cell is a cell from a murine CT26 tumor of colorectal origin. Other specific, non-limiting examples of tumor cells include breast cancer cell lines (for example, 4T1) and sarcoma cell lines (for example, 15-12RM). Cells from excised tumor tissue can be used directly, or the cells can be cultured and expanded under standard culture conditions. Immunogenic whole cells can also be obtained from donor tumor cells that are substantially similar to the subject's tumor. Such donor tumor cells can be obtained, for example, from a donor having a tumor that is the same or substantially similar to the subject's tumor and subsequently inactivating the tumor cell to prevent the cell from multiplying in the subject.

Immunogenic whole cells can be inactivated by methods known in the art. In one embodiment, the cells are irradiated. In other embodiments, the cells are inactivated via oxygen deprivation, use of plant and animal toxins, and chemotherapeutic agents. In yet other embodiments, cells are inactivated with a chemical, such as mitomycin C.

The disclosed methods also use cells that are genetically modified to express an immunogenic agent. Genetically modifying a tumor cell to express an immunogenic agent, such as a known tumor antigen, can be useful when the tumor cells to be administered to a subject to be treated are not obtained from that subject. Donor tumor cells, which may not express one or more particular tumor antigens that are known to be expressed by the subject's tumor cells, can be obtained and can be genetically modified to express the particular tumor antigen, such as E7₍₄₉₋₅₇₎.

Also provided by the disclosure are methods of using dendritic cells (DCs). Upon antigen uptake, DCs residing in peripheral tissues internalize and process antigen and migrate to secondary lymphoid organs where they stimulate naïve T lymphocytes. DCs may be pulsed with an immunogenic agent, for example a tumor peptide antigen (for instance, E7₍₄₉₋₅₇₎) in order to induce an immune response. DCs may also be fused with whole tumor-derived material (for example, live tumor cells or tumor lysates) in order to induce an immune response. In one embodiment, tumor antigen-pulsed DCs, or tumor cell fused DCs, are effective in inducing CTL responses. In other embodiments, tumor antigen-pulsed DCs, or tumor cell fused DCs, are effective at preventing further growth of an existing tumor, enhancing tumor regression, inhibiting tumor recurrence, inhibiting tumor metastasis, or providing protection against subsequent tumor challenge.

### Enhancing an Activity of an Immune Cell by Administering an Immunogenic Agent

The disclosure provides methods of enhancing the activity of an immune cell by administering a combination of agents, wherein one agent is an immunogenic agent, such as a tumor peptide antigen or an inactivated whole cell, thereby affecting tumor growth in a subject.

Immune cells include leukocytes (for instance, neutrophils, eosinophils, monocytes, basophils, macrophages, B cells, T cells, dendritic cells, and mast cells), as well as other types of cells involved in an immune response. The disclosed method includes contacting an immune cell, for example an antigen presenting cell (APC), with a combination of agents including an immunogenic antigen. APCs present antigens to native T cells during the recognition phase of immune responses to initiate these responses and also present antigens to differentiated effector T cells during the effector phase to trigger the mechanisms that eliminate the antigens. In one embodiment, the immune cell is a lymphocyte, such as a T cell or a B cell. In other embodiments, the immune cell is a CTL, a CD8⁺ CTL, a CD4⁺ T cell, a CD4⁺ CD1d-restricted T cell, an NK cell, an NKT cell, or y6 T cells. In a further embodiment, the immune cell is a granulocyte. The immune cell can be either in vivo or in vitro.

In one embodiment, the activity of an immune cell, such a CTL, is enhanced in a subject, following the administration of a combination of agents including an immunogenic agent. For example, the enhanced activity of a CTL may be increased tumor immunosurveillance following the administration of the combination of agents. Another contemplated enhanced immune activity is CD4⁺ T cell activity, which is important to induce good CTL response, NK cell activity, antibody production of B cells and tumordicidal activity of macrophage may also be enhanced. In another embodiment, an enhanced activity of an immune cell affects tumors by enhancing anti-tumor immunity in a subject. In specific embodiments, the enhanced activity of an immune cell prevents further growth of an existing tumor, promotes tumor regression, inhibits tumor recurrence, or inhibits tumor metastasis.

### Enhancing an Immune Response in a Subject by Administering an Immunogenic Agent

The disclosure provides methods of enhancing an immune response in a subject by administering a combination of agents, wherein one agent is an immunogenic agent, such as a tumor peptide antigen or an inactivated whole cell. In one embodiment, an enhanced immune response, for example increased tumor immunosurveillance, enhances the anti-tumor immunity of a subject, thereby affecting tumor growth in the subject.

The disclosed method includes administering to the subject a therapeutically effective amount of a combination of agents in order to enhance an immune response and affect tumors, wherein one of the agents is an immunogenic agent. In one embodiment, the immune response is a T cell response. In a further embodiment, the immune response is CTL-mediated immunosurveillance. In one embodiment, a subject with an enhanced immune response is suffering from a tumor that has escaped CTL immunosurveillance. In another embodiment, an enhanced immune response prevents further growth of an existing tumor, promotes tumor regression, inhibits tumor recurrence, or inhibits tumor metastasis in a subject.

A method is also disclosed herein for enhancing a T cell-mediated immune response. The method includes administering to the subject a therapeutically effective amount of a combination of agents to improve a T cell-mediated immune response, wherein one of the agents is an immunogenic agent. In one embodiment, the T cell-mediated immune response is CTL-mediated immunosurveillance. In another embodiment, the T cell-mediated immune response is an NKT cell response. In a further embodiment, T cell-mediated immune response is a CD4⁺ CD1d-restricted T cell response.

Methods are also provided herein for enhancing a T cell-mediated immune response, such as for instance a CD4 T cell-mediated immune response. Such methods include administering to the subject a therapeutically effective amount of a combination of agents to improve a T cell-mediated immune response, wherein one of the agents is an immunogenic agent. In one embodiment, the T cell-mediated immune response is CTL-mediated immunosurveillance. In another embodiment, the T cell-mediated immune response involves an NKT cell response. In other, embodiments, the response is a CD4 T cell-mediated immune response. In a further embodiment, T cell-mediated immune response is a CD4⁺ CD1d-restricted T cell response.

It is also contemplated that methods provided herein are useful for enhancing anti-viral immunity, for instance, immunity to viruses that cause tumors (e.g., HPV, EBV, and HCV). Such methods involve providing an agent (or combination of agents) that block a TGF-β signaling pathway. In representative examples of such agents, the agent includes a peptide immunogenic agent, such as a peptide vaccine.

### Synergistically Enhancing an Immune Response in a Subject

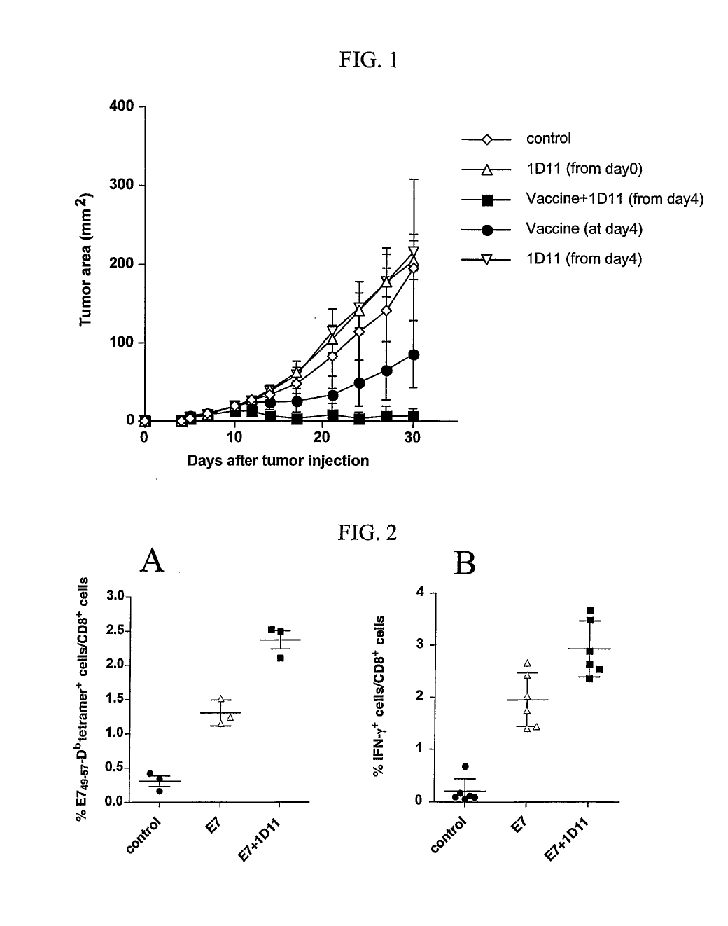

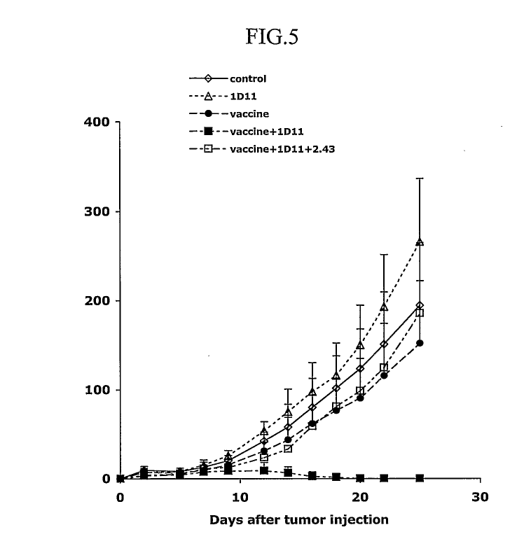

Methods are disclosed herein of enhancing an anti-tumor immunity in a subject by administering a combination of agents, wherein the combination of agents produces a synergistic response that affects tumors, for example preventing further growth of an existing tumor, promoting tumor regression, inhibiting tumor recurrence, or inhibiting tumor metastasis. The disclosed method of administering two or more agents to a subject is more effective than the administration of each agent individually, or the sum of their individual effects. This is illustrated, for instance, in Examples 1 and 4 and in FIGS. 1 and 4. In one embodiment, the administration of an agent that blocks the TGF-β signaling pathway (TGF-p neutralizing agent) enhances the effect of the immunogenic agent on inhibiting, preventing or reversing tumor growth. In another embodiment, the immunogenic agent enhances the effect of the TGF-β neutralizing agent on inhibiting, preventing or reversing tumor growth.

The synergistic combination of agents includes a first agent, such as an immunogenic agent that induces or enhances an immune response. The immunogenic agent can be any tumor antigen, including, but not limited to, inactivated whole tumor cells, lysed tumor cells, and antigenic portions of the tumor cells (for example polypeptides, peptides, membranes, etc.). The synergistic combination also includes a second agent that blocks the TGF-β signaling pathway. The agent can be any agent that blocks TGF-β's immunosuppressive effects, including, but not limited to, an antagonist, an antibody, a neutralizing agent, a chemical compound, a small molecule, a peptide mimetic, an enzyme, a peptide or a protein. One specific, non-limiting example of a combination of agents that generates a synergistic enhancement of tumor regression, compared to each agent individually, or compared to the sum of their individual effects, is the 1D11.16 anti-TGF-β monoclonal antibody in combination with irradiated CT26 cells. Another specific, non-limiting example of a combination of agents that generates a synergistic enhancement of tumor regression is the 1D11.16 anti-TGF-β monoclonal antibody in combination with the E7₍₄₉₋₅₇₎ peptide.

In order to synergistically enhance an immune response in a subject, one or more of immunogenic agents is combined with a pharmaceutically acceptable carrier or vehicle for administration as an immunostimulatory composition or a vaccine (to human or animal subjects). In some embodiments, more than one immunogenic agent may be combined with a pharmaceutically acceptable carrier or vehicle to form a single preparation. In the combination therapy methods, the immunostimulatory composition may be provided to the subject simultaneously with or sequentially with (either before or after) the administration of an agent that that blocks TGF-β's signaling pathway. The immunostimulatory composition and the agent that blocks TGF-β's signaling pathway may be provided prophylactically, for instance prior to detection of a tumor, or prior to the recurrence or metastasis of a tumor in a subject. Alternatively, the immunostimulatory composition and the agent that blocks TGF-β's signaling pathway may be provided therapeutically, for instance in response to the detection of a tumor, in order to prevent further growth of an existing tumor, to promote tumor regression, or to inhibit tumor metastasis. In some embodiments, the immunostimulatory composition may be provided prophylactically and the agent that blocks TGF-β's signaling pathway may be provided therapeutically, or vice versa.

It is also contemplated that the provided immunostimulatory composition and agent that blocks TGF-β's signaling pathway can be administered to a subject indirectly, by first stimulating a cell in vitro, which stimulated cell is thereafter administered to the subject to elicit a synergistic immune response.

### V. Immunological and Pharmaceutical Compositions

The combinations of agents described herein are useful for synergistically enhancing an immune response. Combinations of agents that affect tumors, including an agent effective at blocking the TGF-β signaling pathway in combination with an immunogenic agent, can be administered directly to the subject for preventing further growth of an existing tumor, enhancing tumor regression, inhibiting tumor recurrence, or inhibiting tumor metastasis. The agents may be provided to the subject as immunological or pharmaceutical compositions. In addition, the agents may be provided to the subject simultaneously or sequentially, in either order.

### Immunological Compositions

Immunological compositions, including immunological elicitor compositions and vaccines, and other compositions containing the immunogenic agents described herein, are useful for enhancing an immune response for preventing further growth of an existing tumor, promoting tumor regression, inhibiting tumor recurrence, or inhibiting tumor metastasis. One or more of the immunogenic agents are formulated and packaged, alone or in combination with adjuvants or other antigens, using methods and materials known to those skilled in the vaccine art. An immunological response of a subject to such an immunological composition may be used therapeutically or prophylactically, and in certain embodiments provides antibody immunity and/or cellular immunity such as that produced by T lymphocytes such as cytotoxic T lymphocytes or CD4⁺ T lymphocytes.

A variety of adjuvants known to one of ordinary skill in the art may be administered in conjunction with the immunogenic agents in the provided immunological composition. Such adjuvants include but are not limited to the following: polymers, co-polymers such as polyoxyethylene-polyoxypropylene copolymers, including block co-polymers; polymer P1005; Freund's complete adjuvant (for animals); Freund's incomplete adjuvant; sorbitan monooleate; squalene; CRL-8300 adjuvant; alum; QS 21, muramyl dipeptide; CpG oligonucleotide motifs and combinations of CpG oligonucleotide motifs; trehalose; bacterial extracts, including mycobacterial extracts; detoxified endotoxins; membrane lipids; or combinations thereof.

The compositions provided herein, including those for use as immunostimulatory agents, may be administered through different routes, such as oral, including buccal and sublingual, rectal, parenteral, aerosol, nasal, intramuscular, subcutaneous, intradermal, and topical. They may be administered in different forms, including but not limited to solutions, emulsions and suspensions, microspheres, particles, microparticles, nanoparticles, and liposomes.

The volume of administration will vary depending on the route of administration. By way of example, intramuscular injections may range from about 0.1 ml to 1.0 ml. Those of ordinary skill in the art will know appropriate volumes for different routes of administration.

The amount of immunogenic agent in each immunological composition dose is selected as an amount that induces an immunoprotective response without significant, adverse side effects. Such amount will vary depending upon which specific immunogen is employed and how it is presented. Doses for human administration of a pharmaceutical composition or a vaccine may be from about 0.01 mg/kg to 10 mg/kg, for instance approximately 1 mg/kg. Based on this range, equivalent dosages for heavier (or lighter) body weights can be determined. The dose may be adjusted to suit the individual to whom the composition is administered, and may vary with age, weight, and metabolism of the individual, as well as the health of the subject. Such determinations are left to the attending physician or another familiar with the subject and/or the specific situation. The immunological composition may additionally contain stabilizers or physiologically acceptable preservatives, such as thimerosal (ethyl(2-mercaptobenzoate-S)mercury sodium salt) (Sigma Chemical Company, St. Louis, Mo.). Following an initial vaccination, subjects may receive one or several booster immunizations, adequately spaced. Booster injections may range from 1 μg to 1 mg, with other embodiments having a range of approximately 10 μg to 750 μg, and still others a range of about 50 μg to 500 μg. Periodic boosters at intervals of 1-5 years, for instance three years, may be desirable to maintain the desired levels of protective immunity.

In a particular embodiment, an immunological composition is packaged in a single dosage for immunization by parenteral (for instance, intramuscular, intradermal or subcutaneous) administration or nasopharyngeal (for instance, intranasal) administration. In certain embodiments, the immunological composition is injected intramuscularly into the deltoid muscle. The immunological composition may be combined with a pharmaceutically acceptable carrier to facilitate administration. The carrier is, for instance, water, or a buffered saline, with or without a preservative. The immunological composition may be lyophilized for resuspension at the time of administration or in solution.

The carrier to which the immunogenic agents may be conjugated may also be a polymeric delayed release system. Synthetic polymers are particularly useful in the formulation of a vaccine to affect the controlled release of antigens.

Microencapsulation of the immunogenic agents will also give a controlled release. A number of factors contribute to the selection of a particular polymer for microencapsulation. The reproducibility of polymer synthesis and the microencapsulation process, the cost of the microencapsulation materials and process, the toxicological profile, the requirements for variable release kinetics and the physicochemical compatibility of the polymer and the antigens are all factors that must be considered. Examples of useful polymers are polycarbonates, polyesters, polyurethanes, polyorthoesters polyamides, poly (d,l-lactide-co-glycolide) (PLGA) and other biodegradable polymers.

The compositions provided herein, including those formulated to serve as immunological compositions, may be stored at temperatures of from about −100° C. to 4° C. They may also be stored in a lyophilized state at different temperatures, including higher temperatures such as room temperature. The preparation may be sterilized through conventional means known to one of ordinary skill in the art. Such means include, but are not limited to filtration, radiation and heat. The preparations also may be combined with bacteriostatic agents, such as thimerosal, to inhibit bacterial growth.

### Pharmaceutical Compositions

Pharmaceutical compositions that include one or more agents, such as the 1D11.16 anti-TGF-β antibody or the GC1008 antibody (or other agents discussed herein or known to those in the art), can be formulated with an appropriate solid or liquid carrier, depending on the particular mode of administration chosen. The pharmaceutically acceptable carriers and excipients useful in this disclosure are conventional. For instance, parenteral formulations usually comprise injectable fluids that are pharmaceutically and physiologically acceptable fluid vehicles such as water, physiological saline, other balanced salt solutions, aqueous dextrose, glycerol or the like. Excipients that can be included are, for instance, other proteins, such as human serum albumin or plasma preparations. If desired, the pharmaceutical composition to be administered can also contain minor amounts of non-toxic auxiliary substances, such as wetting or emulsifying agents, preservatives, and pH buffering agents and the like, for example sodium acetate or sorbitan monolaurate.

The dosage form of the pharmaceutical composition will be determined by the mode of administration chosen. For instance, in addition to injectable fluids, topical and oral formulations can be employed. Topical preparations can include eye drops, ointments, sprays and the like. Oral formulations can be liquid (for example, syrups, solutions or suspensions), or solid (for example, powders, pills, tablets, or capsules). For solid compositions, conventional non-toxic solid carriers can include pharmaceutical grades of mannitol, lactose, starch, or magnesium stearate. Actual methods of preparing such dosage forms are known, or will be apparent, to those skilled in the art.

The agents of this disclosure can be administered to humans or other animals on whose cells they are effective in various manners such as topically, orally, intravenously, intramuscularly, intraperitoneally, intranasally, intradermally, intrathecally, and subcutaneously. The particular mode of administration and the dosage regimen will be selected by the attending clinician, taking into account the particulars of the case (for example, the subject, the disease, the disease state involved, and whether the treatment is prophylactic). Treatment can involve daily or multi-daily doses of compound(s) over a period of a few days to months, or even years.

The pharmaceutical compositions that comprise an agent, such as the 1D11.16 or GC1008 anti-TGF-β neutralizing monoclonal antibodies and other agents effective at blocking the TGF-β signaling pathway, in some embodiments of the disclosure will be formulated in unit dosage form, suitable for individual administration of precise dosages. For example, a therapeutically effective amount of the 1D11.16 (or GC1008) anti-TGF-β neutralizing monoclonal antibody can vary from about 0.1 mg/Kg body weight to about 50 mg/Kg body weight. In one specific, non-limiting example, a therapeutically effective amount of the neutralizing monoclonal antibody can vary from about 0.5 mg/Kg body weight to about 25 mg/Kg body weight. In yet another specific, non-limiting example, a therapeutically effective amount of the neutralizing monoclonal antibody can vary from about 1.0 mg/Kg body weight to about 15 mg/Kg body weight. In a further specific, non-limiting example, a therapeutically effective amount of the neutralizing monoclonal antibody can vary from about 5.0 mg/Kg body weight to about 10 mg/Kg body weight.

An effective amount of an agent can be administered in a single dose, or in several doses, for example daily, during a course of treatment. The amount of active compound(s) administered will be dependent on the agent being used, the subject being treated, the severity of the affliction, and the manner of administration, and is best left to the judgment of the prescribing clinician. An effective amount of an agent can be administered prior to, simultaneously with, or following treatment of a tumor. Within these bounds, the formulation to be administered will contain a quantity of the active component(s) in amounts effective to achieve the desired effect in the subject being treated, for instance to measurably reduce the recurrence of a tumor.

A therapeutically effective amount of an agent, such as a neutralizing monoclonal antibody (for example, 1D11.16 or GC1008), can be the amount of agent necessary to inhibit the recurrence of a tumor or the amount necessary to measurably reduce the recurrence of a tumor. In some embodiments, a tumor suppressive amount of an agent is an amount sufficient to inhibit or reduce the recurrence of a tumor (for instance, any of the tumor suppressive amounts discussed herein) without causing a substantial cytotoxic effect (for example, without killing more than 1%, 2%, 3%, 5%, or 10% of normal cells in a sample).

Site-specific administration of the disclosed compounds can be used, for instance by applying an agent, such as the 1D11.16 or GC1008 anti-TGF-β neutralizing monoclonal antibody, to a region of tissue from which a tumor has been removed or near a region of tissue from which a tumor has been removed. In some embodiments, sustained intra-tumoral (or near-tumoral) release of the pharmaceutical preparation that comprises a therapeutically effective amount of an agent, such as the 1D11.16 or GC1008 anti-TGF-β neutralizing monoclonal antibody, may be beneficial. Slow-release formulations are known to those of ordinary skill in the art. By way of example, polymers such as bis(p-carboxyphenoxy)propane-sebacic-acid or lecithin suspensions may be used to provide sustained intra-tumoral release.

It is specifically contemplated in some embodiments that delivery is via an injected and/or implanted drug depot, for instance comprising multi-vesicular liposomes such as in DepoFoam (SkyePharma, Inc, San Diego, Calif.) (see, for instance, Chamberlain et al., Arch. Neuro. 50:261-264, 1993; Katri et al., J. Pharm. Sci. 87:1341-1346, 1998; Ye et al., J. Control Release 64:155-166, 2000; and Howell, Cancer J 7:219-227, 2001).

### Combined Compositions

A pharmaceutical composition, described above, can be combined with an immunological composition, described above, in order to administer a combination of agents in a single dose. It is contemplated that an immunological composition including an immunogenic agent, such as a tumor peptide antigen or an inactivated whole cell, be combined with a pharmaceutical composition including an agent that blocks TGF-β signaling. In one embodiment, a composition including a neutralizing anti-TGF-β monoclonal antibody (for example, 1D11.16 or GC1008) and a E7₍₄₉₋₅₇₎ peptide mixed together is administered to a subject as a single dose. In another embodiment, a composition including a neutralizing anti-TGF-β monoclonal antibody (for example, 1D11.16 or GC1008) and irradiated CT26 cells are mixed and administered to a subject as a single dose. As discussed above, the dose of the composition, the route of administration, and the frequency and the rate of administration will vary. Examples and guidelines for dosing are described above; yet more will be known to those of ordinary skill in the art.

Aspects are further illustrated by the following non-limiting Examples.

### EXAMPLES

### Example 1

### Blockade of TGF-β Synergistically Enhances Peptide Vaccine Efficiency in Mice

It was previously demonstrated that a negative immunoregulatory pathway suppresses CTL-mediated anti-tumor immunity in tumor-bearing animals. In this pathway TGF-β produced by myeloid cells is induced by interleukin (IL)-13, which is made by NKT cells. This TGF-β is the final effector molecule to suppress CTL activation. In addition, it was demonstrated previously that blocking this TGF-p enhanced spontaneous tumor immunosurveillance, led to tumor rejection in several mouse tumor models. However, this blockade is not always sufficient to induce tumor rejection. Therefore, the effect of blocking TGF-β, using an anti-TGF-β antibody (1D11.16), on the efficacy of therapeutic anti-tumor peptide vaccines in mice was examined.

TC1 is a C57BL/6-derived lung epithelial cell line transfected with the E6 and E7 genes of Human Papilloma Virus (HPV)-16, along with mutant ras. The cells were maintained in RPMI 1640 medium containing 10% fetal calf serum, L-glutamine, sodium pyruvate, nonessential amino acids, penicillin, streptomycin, and 5×10⁻⁵ M 2-mercaptoethanol, containing 200 μg/ml of geneticin.

Syngeneic C57BL/6 mice were challenged with TC1 cells subcutaneously by inoculating the mice subcutaneously with 2×10⁴ TC1 cells suspended in Hanks' balanced buffer solution into the right flank. After 4-8 days, when palpable tumors were well established, some mice were immunized subcutaneously with 100 μg of Human Papilloma Virus (HPV) E7₍₄₉₋₅₇₎ peptide emulsified in 100 μl of incomplete Freund's adjuvant with a hepatitis B virus (HBV) core₍₁₂₈₋₁₄₀₎ helper epitope peptide (10 nmol) and granulocyte-macrophage colony stimulating factor (GM-CSF; 10 μg). Some mice were injected with 100 μg of anti-TGF-β monoclonal antibody (1D11.16) or control antibody (13C4) intraperitoneally three times a week from the day of tumor inoculation, or from the time of vaccination, until the end of the experiment (three weeks). Tumors were measured by a caliper gage, and tumor size was determined as the product of tumor length (mm)×tumor width (mm). Five female C57BL/6 mice were used for each group.

The treatment with 1D11.16 alone (without vaccine) did not show any effect on tumor growth. The tumors in the group of mice treated with vaccine alone showed a significant delay of tumor growth, compared to the tumors in untreated mice, but none of the tumors regressed. The mice treated with both vaccine and 1D11.16 showed either partial regression or complete rejection of the tumors. These results indicated that the combination of the 1D11.16 antibody and a peptide vaccine (E7₍₄₉₋₅₇₎; SEQ ID NO: 1) synergistically enhanced anti-tumor immunity in a therapeutic setting (FIG. 1).

### Example 2

### Blockade of TGF-β Synergistically Enhances Peptide Vaccine Efficiency to induce tumor antigen-specific CD8⁺ CTLs in Mice

This experiment was performed to determine if blockade of TGF-β enhances efficacy of the HPV E7₍₄₉₋₅₇₎ peptide vaccine to induce tumor antigen-specific CD8⁺ cytotoxic T lymphocytes (CTLs) in tumor-bearing individuals. C57BL/6 mice were inoculated subcutaneously with 2×10⁴ TC1 cells. On day seven, some mice were immunized subcutaneously with 100 μg of HPV E7₍₄₉₋₅₇₎ peptide emulsified in incomplete Freund's adjuvant with a HBV core helper epitope peptide (50 nmol) and GM-CSF (5 μg). Some mice were injected with 100 μg of anti-TGF-β monoclonal antibody (1D11.16) intraperitoneally three times a week from day 4 to day 21. Five mice were used for each group. Two weeks after immunization, the mice were euthanized and spleen cells were examined for a specific response against HPV E7₍₄₉₋₅₇₎.

To measure the number of HPV E7₍₄₉₋₅₇₎-specific CD8⁺ T cells, spleen cells were stained with D b-tetramer loaded with HPV E7₍₄₉₋₅₇₎ peptide along with anti-mouse CD8 antibody, and measured by flow cytometry. For measurement of HPV E7₍₄₉₋₅₇₎-specific IFN-γ producing response of CD8⁺ T cells, the cells were cultured with T cell-depleted naïve spleen cells pulsed with/without 0.1 μM of HPV E7₍₄₉₋₅₇₎ overnight. Then the cells were stained for surface CD8 and intracellular IFN-γ, and measured by flow cytometry. To measure in vivo tumor-antigen specific lytic activity, an in-vivo CTL assay was performed. Thirteen days after immunization of TC1-challenged mice, a 1:1 mixture of spleen cells (1×10⁷ of each) of naïve mice pulsed with or without 0.1 μM of HPV E7₍₄₉₋₅₇₎ and labeled with different concentrations of CFSE was injected intravenously. The next day, spleen cells from the mice were harvested and residual CFSE cells were measured by flow cytometry. The proportion of the cells with different CFSE brightness was determined, and compared with the proportion in naïve cells that received the same cells to compute HPV E7₍₄₉₋₅₇₎-specific lytic activity.

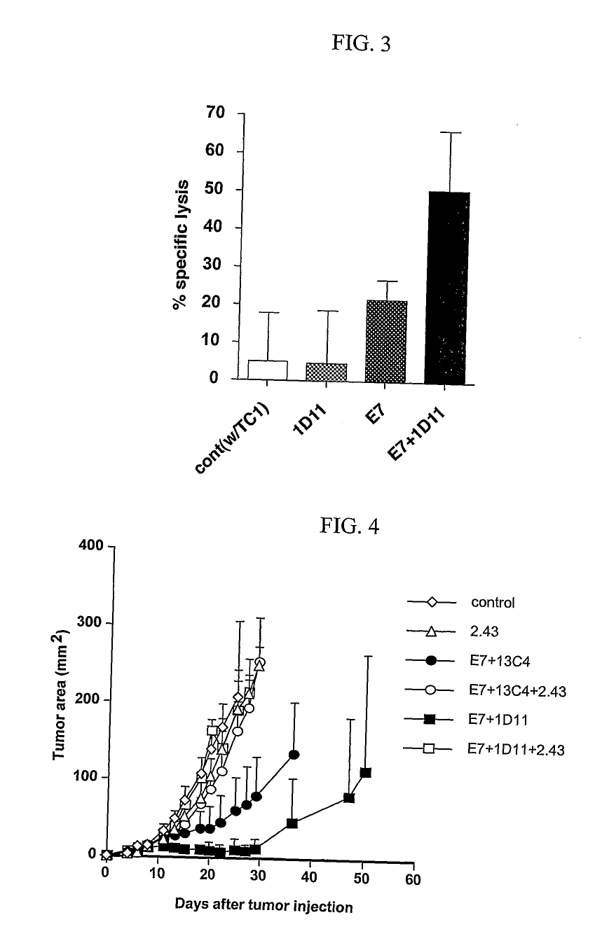

The mice that received HPV E7₍₄₉₋₅₇₎ peptide vaccine alone had a significantly higher frequency of HPV E7₍₄₉₋₅₇₎-specific CD8⁺ T cells (FIG. 2A), HPV E7₍₄₉₋₅₇₎-specific IFN-γ production response (FIG. 2B) and in vivo lytic activity against HPV E7₍₄₉₋₅₇₎ pulsed target cells (FIG. 3). However, combination treatment with both vaccine and 1D11.16 induced significantly enhanced HPV E7₍₄₉₋₅₇₎-specific CD8 T cell responses (FIGS. 2A and 2B and FIG. 3). These results strongly indicate that the combination of the 1D11.16 antibody and a peptide vaccine (E7₍₄₉₋₅₇₎; SEQ ID NO: 1) synergistically enhanced anti-tumor CD8+ T cell-responses that may be critical for anti-tumor immunity.

### Example 3

### Anti-CD8 Antibody Completely Abrogates Protection in Vaccinated Mice

This experiment was performed to determine if protection induced by the HPV E7₍₄₉₋₅₇₎ peptide vaccine is mediated by CD8⁺ cytotoxic T lymphocytes (CTLs). C57BL/6 mice were inoculated subcutaneously with 2×10⁴ TC1 cells. On day 7, some mice were immunized subcutaneously with 100 μg of HPV E7₍₄₉₋₅₇₎ peptide emulsified in incomplete Freund's adjuvant with a HBV core helper epitope peptide (50 nmol) and GM-CSF (5 μg). Some mice were injected with 100 μg of anti-TGF-β monoclonal antibody (1D11.16) intraperitoneally three times a week from day 7 to day 21 or with a control antibody 13C4. Some mice were also treated intraperitoneally with 0.5 mg of anti-CD8 monoclonal antibody (2.43) on days 7, 8, 13, 15, 20. Alternatively, the mice were treated intraperitoneally three days in a row and then once a week. Five mice were used for each group.

Anti-CD8 antibody treatment completely abrogated the protection in vaccinated mice (FIG. 4). These results indicate that the protection induced by the vaccine was CD8⁺ CTL mediated. Taken together, these results clearly indicated that blockade of TGF-β synergistically enhances anti-tumor immunity in conjunction with therapeutic administration of a tumor peptide vaccine.

### Example 4

### Blockade of TGF-β Synergistically Enhances Whole Cell Vaccine in Mice

The effect of blocking TGF-β, using an anti-TGF-β antibody (1D11.16), on the efficacy of prophylactic anti-tumor whole cell vaccines in mice was examined.

The CT26 cell line (a N-nitro-N-methylurethane-induced BALB/c murine colon carcinoma) was maintained in RPMI 1640 medium containing 10% fetal calf serum, L-glutamine, sodium pyruvate, nonessential amino acids, penicillin, streptomycin, and 5×10⁻⁵ M 2-mercaptoethanol, containing 200 μg/ml of geneticin. The cells were washed and suspended in PBS prior to injections.

For immunizations, the cells were harvested and irradiated with 25,000 rad. Irradiated CT26 cells (a colon carcinoma cell line derived from a BALB/c mouse) was administered prophylactically by subcutaneous injection to syngeneic BALB/c mice. The whole tumor cell vaccine (irradiated CT26 cells) alone induced a significant delay of tumor growth, compared to control mice and the mice treated with 1D11.16 alone. However, none of the mice that received the vaccine alone were protected from tumors. In contrast, surprisingly the vaccine in combination with 1D11.16 induced complete tumor regression, even though palpable tumors appeared at first after the tumor challenge. Taken together, these results clearly indicated that blockade of TGF-β synergistically enhances anti-tumor immunity in conjunction with prophylactic administration of a whole cell tumor vaccine (FIG. 5).

This disclosure provides, in various embodiments, methods of inhibiting tumor growth. The disclosure further provides combinations of agents that synergistically enhance tumor regression. It will be apparent that the precise details of the methods described may be varied or modified without departing from the spirit of the described invention. We claim all such modifications and variations that fall within the scope and spirit of the claims below.

We claim:

1. A method of enhancing tumor regression in a subject, comprising:
administering to the subject (1) a therapeutically effective amount of an antibody, wherein the antibody inhibits transforming growth factor (TGF)-p activity in the subject, and (2) an immunogenic agent, wherein the agent is a tumor vaccine, such as a tumor peptide, or an inactivated whole cell, wherein the subject has a tumor or is at risk of developing a tumor, thereby enhancing tumor regression in the subject.

2. The method of claim 1, wherein the antibody is a polyclonal antibody or a monoclonal antibody.

3. The method of claim 2, wherein the antibody is specific for a TGF-β.

4. The method of claim 3, wherein the anti-TGF-β antibody inhibits TGF-p from binding a TGF-β receptor.

5. The method of claim 2, wherein the monoclonal antibody is obtained from hybridoma 1D11.16 (ATCC Accession No. HB 9849) or GC1008, or is a humanized version of the monoclonal antibody.

6. The method of claim 1, wherein the tumor peptide is a Human Papilloma Virus (HPV)-16 peptide.

7. The method of claim 6, wherein the HPV peptide is an E6 or an E7 peptide.

8. The method of claim 7, wherein the E7 peptide is the E7₍₄₉₋₅₇₎ peptide epitope.

9. The method of claim 1, wherein the inactivated whole cell is an irradiated cell.

10. The method of claim 1, wherein the inactivated whole cell is an irradiated CT26 murine colorectal tumor cell.

11. The method of claim 1, wherein the subject is a human.

12. The method of claim 1, wherein the tumor is benign or malignant.

13. The method of claim 1, wherein the tumor is a primary tumor or a metastasis.

14. The method of claim 1, wherein the tumor comprises a carcinoma, a sarcoma, a leukemia, or a tumor of the nervous system.

15. The method of claim 1, wherein the tumor comprises a breast tumor, a liver tumor, a pancreatic tumor, a gastrointestinal tumor, a colon tumor a uterine tumor, a ovarian tumor, a cervical tumor, a testicular tumor, a brain tumor, a skin tumor, a melanoma, a retinal tumor, a lung tumor, a kidney tumor, a bone tumor, a prostate tumor, a nasopharyngeal tumor, a thyroid tumor, a leukemia, or a lymphoma.

16. The method of claim 1, wherein administering to the subject comprises intravenous, subcutaneous, intradermal, or intramuscular administration, or any combination thereof.

17. The method of claim 1, wherein administering to the subject comprises administration prior to detection of the tumor or following detection of the tumor.

18. The method of claim 1, wherein inhibiting TGF-β blocks an immunosuppressive effect in the subject.

19. The method of claim 1, wherein inhibiting TGF-β comprises increased immunosurveillance by lymphocytes of the subject.


SEQUENCE LISTING (6 sequences)

SEQ ID NO: 1 - protein, 9 aa, Human papillomavirus
RAHYNIVTF

SEQ ID NO: 2 - protein, 98 aa, Human papillomavirus
MHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKP

SEQ ID NO: 3 - protein, 9 aa, Artificial sequence
  peptide
SPSYVYHQF

SEQ ID NO: 4 - protein, 8 aa, Artificial
  peptide
ITQVPFSV

SEQ ID NO: 5 - protein, 8 aa, Artificial
  peptide
FLRNFSLM

SEQ ID NO: 6 - protein, 9 aa, Artificial
  peptide
FVFLRNFSL